(19) United States
(12) Patent Application Publication
(10) Pub. No.: US 2011/0262465 A1
(43) Pub. Date: Oct. 27, 2011

(54) PROSTATE STEM CELLS AND USES THEREOF

(75) Inventors: Wei-Qiang Gao, Palo Alto, CA (US); Leisa Johnson, Point Richmond, CA (US); Kevin G. Leong, Burlingame, CA (US)

(73) Assignee: Genentech, Inc., South San Francisco, CA (US)

(21) Appl. No.: 13/124,428

(22) PCT Filed: Oct. 21, 2009
(86) PCT No.: PCT/US2009/061473

§ 371 Date: Jul. 7, 2011

Related U.S. Application Data
(60) Provisional application No. 61/196,930, filed on Oct. 22, 2008.

(51) Int. Cl. A61K 39/395; C12N 5/02; A61P 35/00; A61K 31/404; C12Q 1/02; C12N 5/00; A61K 31/497

(52) U.S. Cl. 424/174.1; 435/325; 435/375; 514/252.18; 514/414; 435/29

(57) ABSTRACT

Prostate stem cells and prostate cancer stem cells and their use in treating prostate cancer and regenerating prostate tissue are disclosed.

### RELATED APPLICATIONS

This application claims the benefit of priority under 35 USC 119(e) of U.S. Provisional Application No. 61/196,930, filed Oct. 22, 2008, the disclosure of which is incorporated herein by reference in its entirety.

### FIELD OF THE INVENTION

The present invention relates to prostate stem cells and prostate cancer stem cells and their use in treating prostate cancer and regenerating prostate tissue.

### BACKGROUND

The existence of prostate stem cells (PSCs) has been proposed based on the observation that normal prostate regeneration can occur following repeated cycles of androgen deprivation and replacement in rodents¹. The prostate is dependent upon androgen for proper growth and tissue homeostasis⁷. Following androgen deprivation, the prostate undergoes involution due to the apoptosis of cells that require androgen for survival. Remarkably, replacement of androgen induces regeneration of the prostate back to its original size and functional state. The fact that the involution/regeneration process can be repeated over 30 cycles in the rodent prostate¹ demonstrates that the adult prostate contains a long-lived, androgen-independent stem cell population. Stem cell-enriched populations have been identified in both the mouse and human prostates^{2-6,8-10}.

Several cell-surface markers have been reported to identify candidate PSCs, including stem cell antigen-1 (Sca-1), CD133 (prominin-1), and CD44²⁻⁶. However, non-PSCs in the mouse prostate also express these markers and thus identification of a defined PSC population remains elusive.

CD117 (c-kit, stem cell factor receptor) is a member of the receptor tyrosine kinase subclass III family and is related to the receptors for platelet-derived growth factor, macrophage colony-stimulating factor, and FMS-like receptor tyrosine kinase (FLT3) ligand. Heinrich, M. C. et al., J. Clin. Oncol. 20 (6): 1692-1703 (2002). c-Kit and its ligand Stem Cell Factor (SCF) are essential for haemopoiesis, melanogenesis and fertility. SCF acts at multiple levels of the haemopoietic heirarchy to promote cell survival, proliferation, differentiation, adhesion and functional activation. Ashman, L. K., et al, Intl. J. of Biochem. Cell Biol. 31:1037-1051 (1999). CD117 expression has been observed in human malignancies and the kinase activity of CD117 has been implicated in the pathophysiology of a number of these tumors, including mastocytosis/mast cell leukemia, germ cell tumors, small-cell lung carcinoma (SCLC), gastrointestinal stromal tumors (GIST), acute myelogenous leukemia (AML), neuroblastoma, melanoma, ovarian carcinoma, and breast carcinoma. J. Clin. Oncol. 20 (6): 1692-1703 (2002) (supra).

Cancer stem cells (CSCs) are cells within a tumor that possess the capacity to self-renew and to give rise to the heterogeneous lineages of cancer cells that comprise the tumor. Clark, M. F. et al., Cancer Res. 66 (19): 9339-9344 (2006). These cells are thought to be responsible for metastasis, therapy resistance, and recurrence. However, CSCs constitute only a small fraction of a cancer tumor mass and are difficult to identify and/or isolate. It is believed that cancer stem cells arise from normal stem cells that have undergone mutation. These mutated stem cells undergo neoplastic transformation to form a tumor that contains cancer stem cells that can be identified by the same markers present on the normal stem cell. Zhu, L. et al., Nature 457, 603-607 (2009).

### SUMMARY OF THE INVENTION

One aspect of the invention provides an isolated prostate stem cell that expresses CD117. In one embodiment, the stem cell further expresses CD133 and CD44. In one embodiment, the stem cell further expresses CD133 and CD44 and Sca-1. In some embodiments, the prostate stem cell is capable of generating lumen-containing prostate colonies in vitro. In other embodiments the prostate stem cell is capable of generating a functional prostate in vivo.

Another aspect of the invention provides an isolated prostate cancer stem cell that expresses CD117. In one embodiment, the prostate cancer stem cell further expresses CD133 and CD44. In another embodiment, the prostate cancer stem cell further expresses CD133 and CD44 and Sca-1. In some embodiments, the prostate cancer stem cell is capable of generating prostate cancer in an in vivo model.

Another aspect of the invention provides for a method of isolating a prostate stem cell comprising obtaining a lineage depleted (Lin−) prostate cell population and sorting the Lin− cell population to obtain a population of cells that expresses CD117, CD133, and CD44. In one embodiment, the method further comprises sorting the Lin− prostate cell population to obtain a population of cells that expresses Sca-1. The cells are sorted, for example, using fluorescence-activated cell sorting (FACS).

Another aspect of the invention provides for a method of inhibiting the proliferation of a prostate stem cell or prostate cancer stem cell that expresses CD117, CD133, and CD44, comprising contacting the prostate stem cell or prostate cancer stem cell with an therapeutically effective amount of a CD117 antagonist.

Another aspect of the invention provides for a method of preventing reoccurrence of prostate cancer in a patient comprising determining if the patient's prostate cancer comprises a prostate cell that expresses CD117, CD133, and CD44, and administering to the patient a therapeutically effective amount of a CD117 antagonist if the patient's prostate cancer comprises a prostate cell that expresses CD117, CD133, and CD44. In one embodiment, an effective amount of a CD133 antagonist or a CD44 antagonist is also administered to the patient.

Another aspect of the invention provides for a method of selecting a prostate cancer patient for treatment with a CD117 antagonist comprising determining if the patient has a prostate cancer that comprises a prostate cell that expresses CD117, CD133, and CD44, and selecting the patient for treatment with a CD117 antagonist if the patient has a prostate cancer that comprises a prostate cell that expresses CD117, CD133, and CD44. In some embodiments, the patient has had a recurrence of the prostate cancer.

Yet another aspect of the invention provides for a method of treating prostate cancer in a patient comprising determining if the patient has a prostate cancer that comprises a prostate cell that expresses CD117, CD133, and CD44, and administering to the patient a therapeutically effective amount of a CD117 antagonist if the patient has a prostate cancer that comprises a prostate cell that expresses CD117, CD133, and CD44. In one embodiment, an effective amount of a CD133 antagonist or a CD44 antagonist is also administered to the patient. In some embodiments, the patient has had a recurrence of the prostate cancer.

Yet another aspect of the invention provides for a method of selecting a prostate cancer patient for adjuvant treatment with a CD117 antagonist comprising determining if the patient's prostate cancer comprises a prostate cell that expresses CD117, CD133, and CD44, and selecting the patient for adjuvant treatment with a CD117 antagonist if the patient's prostate cancer comprises a prostate cell that expresses CD117, CD133, and CD44.

Yet another aspect of the invention provides for a method of providing adjuvant therapy to a patient treated for prostate cancer comprising determining if the patient's prostate cancer comprises a prostate cell that expresses CD117, CD133, and CD44, and administering to the patient a therapeutically effective amount of a CD117 antagonist if the patient's prostate cancer comprises a prostate cell that expresses CD117, CD133, and CD44. In one embodiment, an effective amount of a CD133 antagonist or a CD44 antagonist is also administered to the patient.

In some embodiments of the above aspects, the CD117 antagonist is an anti-CD117 antibody, or a small molecule, such as imatinib mesylate or sunitinib malate.

A further aspect of the invention provides for a method of promoting prostate tissue growth or repair comprising implanting a prostate stem cell which expresses CD117, CD133, and CD44 in a patient in need of prostate tissue growth or repair. In one embodiment, the patient has a partial prostate and the stem cell is implanted in the partial prostate.

A still further aspect of the invention provides for a method of promoting prostate growth comprising implanting a prostate stem cell which expresses CD117, CD133, and CD44 in a mammalian host under conditions to generate a functioning prostate. In one embodiment, the stem cell is implanted under the renal capsule of the host. In one embodiment, the host is a human, in another embodiment the host is a pig.

A further aspect of the invention provides for a method of providing a functional prostate to a patient in need thereof comprising implanting a human prostate stem cell which expresses CD117, CD133, and CD44 in a mammalian host under conditions to generate a functioning prostate and harvesting the prostate. In one embodiment, the harvested prostate is transplanted into a human patient in need of a functioning prostate.

Another aspect of the invention provides for a method of screening for a compound that inhibits the proliferation of prostate cancer stem cells comprising contacting a prostate cancer stem cell that expresses CD117, CD133, and CD44 with a test compound, and detecting whether the test compound inhibits proliferation of the cell.

### BRIEF DESCRIPTION OF THE DRAWINGS

FIG. 1 shows an intact prostate from an adult C57BL/6 mouse. Four pairs of lobes (D—dorsal, L—lateral, V—ventral, A—anterior) are shown. Bottom panel indicates distal, intermediate, and proximal regions for each prostatic lobe, relative to the urethra.

FIG. 2 is a graph showing Q-RT-PCR for gene expression in the different regions of the adult C57BL/6 prostate, normalized to the distal region. Statistical comparisons with distal: *P<0.05; **P<0.01; ***P<0.001.

FIG. 3a is a graph showing Q-RT-PCR for gene expression in the different lobes of the adult C57BL/6 prostate, normalized to the dorsal lobe. FIG. 3b is a statistical analysis of the gene expression among dorsal (D), lateral (L), ventral (V), and anterior (A) lobes. *P<0.05; **P<0.01; ***P<0.001; ****P<0.0001.

FIG. 4 is a Q-RT-PCR analysis of gene expression in adult C57BL/6 prostates (normal, 3 days post-castration, 14 days post-castration, and 14 days post-castration with 3 days hormone replacement). Data are expressed as fold change relative to GADPH. Statistical comparisons with hormone replacement: *P<0.05; **P<0.01; ***P<0.0001. All bars represent the mean+s.e.m.

FIG. 5 is a graph showing enrichment of CD117+ cells using magnetic bead sorting.

FIG. 6 is a schematic diagram of the serial isolation and transplantation procedure used verify that CD117+ prostate stem cells have self-renewal capacity.

FIG. 7a is a graph showing quantification of the net growth in prostate area over time for prostates treated with an anti-CD117 antibody and for untreated prostates. *P<0.05; **P<0.01.

FIG. 7b is a graph showing quantification of the branch points per mm² for prostates as determined on day 8 of the anti-CD117 treatment and for untreated prostates. *P<0.001.

FIG. 8a is a Q-RT-PCR analysis for SLUG expression in CD117+/− sorted populations. *P<0.03. FIG. 8b is a microarray analysis of SLUG expression in adult C57BL/6 prostates (normal, 3 days post-castration, 14 days post-castration, and 14 days post-castration with 3 days hormone replacement). Data are expressed as fold change relative to normal. Statistical comparisons with hormone replacement: *P<0.02. FIG. 8c is a Q-RT-PCR analysis for SLUG expression in ex vivo prostates treated with an anti-CD117 antibody. Data are expressed as fold change relative to day 1 control. *P<0.003.

FIG. 9 is a graph showing the percentage of viable cells within the adult C57BL/6 prostate expressing single and multiple markers of prostate stem cells.

FIG. 10 is a flow diagram of the fluorescence-activated cell sorting procedure used to obtain cell populations expressing combinations of the surface markers Sca-1, CD133, CD44, and CD117.

FIG. 11 is a graph showing quantification of graft weight three months post renal capsule implantation. Data are from two independent experiments. *(Lin⁻Sca-1⁺CD133⁺CD44⁺CD117⁺ versus Lin⁻ Sca-1⁻CD133⁻CD44⁻CD117⁻: P=0.002; Lin⁻Sca-1⁺CD133⁺CD44⁺CD117⁺ versus Lin⁻Sca-1⁺CD133⁺CD44⁺CD117⁻: P=0.03; Lin⁻Sca-1⁺CD133⁺CD44⁺CD117⁺ versus UGM only: P=0.004).

FIG. 12 is a graph showing the prostate generation capacities of UGM only, Lin-Sca-1-CD133−CD44−CD117−, Lin-Sca-1+CD133+CD44+CD117+, and Lin-Sca-1+CD133+CD44+CD117− implants three months post renal capsule implantation.

FIG. 13 is a schematic diagram of the single cell transplantation procedure.

FIG. 14a shows PCR-based genotyping following laser capture microdissection (LCM) of cells isolated from single cell implant. FIG. 14b shows a limiting dilution analysis to determine the frequency of prostate stem cells within the Lin-Sca-1+CD133+CD44+CD117+ cell population.

FIG. 15a is a graph showing the percentage of viable cells within human clinical benign non-BPH prostate specimens expressing single and multiple markers of prostate stem cells. FIG. 15b is a graph showing the percentage of viable cells within human clinical BPH prostate specimens expressing single and multiple markers of prostate stem cells.

### DETAILED DESCRIPTION OF THE PREFERRED EMBODIMENTS

### Definitions

The term “CD117” refers to any CD117 from any vertebrate source, including mammals such as primates (e.g. humans and monkeys) and rodents (e.g., mice and rats), unless otherwise indicated. The term encompasses “full-length,” unprocessed CD117 as well as any form of CD117 that results from processing in the cell. The term also encompasses naturally occurring variants of CD117 e.g., splice variants, allelic variants, and other isoforms. The term also encompasses fragments or variants of a native CD117 that maintain at least one biological activity of CD117, e.g., kinase activity. CD117 is also referred to in the scientific literature as c-kit and as stem cell factor receptor.

The term “CD44” refers to any CD44 from any vertebrate source, including mammals such as primates (e.g. humans and monkeys) and rodents (e.g., mice and rats), unless otherwise indicated. The term encompasses “full-length,” unprocessed CD44 as well as any form of CD44 that results from processing in the cell. The term also encompasses naturally occurring variants of CD44 e.g., splice variants, allelic variants, and other isoforms. The term also encompasses fragments or variants of a native CD44 that maintain at least one biological activity of CD44.

The term “CD133” refers to any CD133 from any vertebrate source, including mammals such as primates (e.g. humans and monkeys) and rodents (e.g., mice and rats), unless otherwise indicated. The term encompasses “full-length,” unprocessed CD133 as well as any form of CD133 that results from processing in the cell. The term also encompasses naturally occurring variants of CD133 e.g., splice variants, allelic variants, and other isoforms. The term also encompasses fragments or variants of a native CD44 that maintain at least one biological activity of CD133. CD133 is also referred to in the scientific literature as prominin-1

The term “Sca-1” refers to any Sca-1 from any vertebrate source, including mammals such as primates (e.g. humans and monkeys) and rodents (e.g., mice and rats), unless otherwise indicated.

The term encompasses “full-length,” unprocessed Sca-1, as well as any form of Sca-1 that results from processing in the cell. The term also encompasses naturally occurring variants of Sca-1 e.g., splice variants, allelic variants, and other isoforms. The term also encompasses fragments or variants of a native Sca-1 that maintain at least one biological activity of Sca-1.

The term “prostate stem cell” (PSC) or “prostate stem cells” (PSCs) or as used herein refers to a prostate cell or prostate cells that can self-renew and are capable of generating all epithelial cell types found within a prostate. Prostate stem cells can be detected by their ability to generate lumen-containing prostate colonies in vitro. Prostate stem cells are ultimately capable of generating a functional prostate in vivo.

The term “prostate cancer stem cell” (PCSC) or “prostate cancer stem cells” (PCSCs) as used herein refers to a cell or cells that can give rise to tumorigenic cells associated with prostate cancer. Prostate cancer stem cells are the cells responsible for establishing prostate cancer from a mutated normal prostate, and/or re-establishing prostate cancer following primary cancer treatment. Prostate cancer stem cells can be detected using in vitro cellular proliferation assays. They can also be detected using in vivo transplantation assays. For example, the proposed prostate cancer stem cells are injected or transplanted in an in vivo host model and the model is examined to determine if injection/transplantation of the cells results in the model developing prostate cancer. Those cells that generate prostate cancer in the model are prostate cancer stem cells.

The term “polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs.

The term “detection” includes any means of detecting, including direct and indirect detection.

The term “diagnosis” is used herein to refer to the identification of a molecular or pathological state, disease or condition, such as the identification of a cancer (e.g., a prostate cancer) or a particular type of cancer (e.g., a prostate cancer characterized by a particular variation). The term “prediction” is used herein to refer to the prediction of the likelihood of cancer-attributable death or progression, including, for example, recurrence, metastatic spread, and drug resistance, of a neoplastic disease, such as cancer. The term “prediction” is also used herein to refer to the likelihood that a patient will respond either favorably or unfavorably to a drug or set of drugs. In one embodiment, the prediction relates to the extent of those responses. In one embodiment, the prediction relates to whether and/or the probability that a patient will survive following treatment, for example treatment with a particular therapeutic agent and/or surgical removal of the primary tumor, and/or chemotherapy for a certain period of time without cancer recurrence. In another embodiment, the prediction relates to whether and/or the probability that a patient experience a reoccurrence of the cancer. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely. Patients may be selected to receive a particular treatment based on the predictive methods of the invention.

The terms “cell proliferative disorder” and “proliferative disorder” refer to disorders that are associated with a measurable degree of abnormal cell proliferation. In one embodiment, the cell proliferative disorder is cancer.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The terms “cancer” and “cancerous” refer to or describe the physiological condition in mammals that is typically characterized by unregulated cell growth and proliferation. Examples of cancer include, but are not limited to, carcinoma, lymphoma (e.g., Hodgkin's and non-Hodgkin's lymphoma), blastoma, sarcoma, and leukemia. More particular examples of cancers include squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer, colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia and other lymphoproliferative disorders, and various types of head and neck cancer.

The term “prostate tumor” or “prostate cancer” refers to any tumor or cancer of the prostate.

The term “prostate tumor cell” or “prostate cancer cell” refers to a prostate tumor cell or prostate cancer cell, either in vivo or in vitro, and encompasses cell lines derived from such cells.

The term “neoplasm” or “neoplastic cell” refers to an abnormal tissue or cell that proliferates more rapidly than corresponding normal tissues or cells and continues to grow after removal of the stimulus that initiated the growth.

A “tumor cell” or “cancer cell” refers to a tumor cell or cancer cell, either in vivo or in vitro, and encompasses cell lines derived from such cells.

As used herein, “treatment” (and variations such as “treat” or “treating”) or “therapy” refers to clinical intervention in an attempt to alter the natural course of the individual or cell being treated, and can be performed either for prophylaxis or during the course of clinical pathology. Desirable effects of treatment include preventing occurrence or reoccurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “adjuvant therapy” refers to the additional cancer treatment given after the primary cancer treatment to lower the risk of cancer reoccurrence.

An “individual,” “subject,” or “patient” is a vertebrate. In certain embodiments, the vertebrate is a mammal. Mammals include, but are not limited to, farm animals (such as pigs and cows), sport animals, pets (such as cats, dogs, and horses), primates (including human and non-human primates), and rodents (e.g., mice and rats). In certain embodiments, a mammal is a human.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects.

The term “long-term” survival is used herein to refer to survival for at least 1 year, 5 years, 8 years, or 10 years following therapeutic treatment.

The term “increased resistance” to a particular therapeutic agent or treatment option, when used in accordance with the invention, means decreased response to a standard dose of the drug or to a standard treatment protocol.

The term “decreased sensitivity” to a particular therapeutic agent or treatment option, when used in accordance with the invention, means decreased response to a standard dose of the agent or to a standard treatment protocol, where decreased response can be compensated for (at least partially) by increasing the dose of agent, or the intensity of treatment.

“Patient response” can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

A tumor or cancer that “responds” to a therapeutic agent is one that shows any decrease in tumor progression, including but not limited to, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; and/or (5) inhibition (i.e. reduction, slowing down or complete stopping) of metastasis.

The term “antagonist” is used in the broadest sense, and includes any molecule that partially or fully inhibits or neutralizes a biological activity (e.g., kinase activity) of a polypeptide (e.g., CD117), or that partially or fully inhibits the transcription or translation of a nucleic acid encoding the polypeptide. Suitable antagonist molecules include, but are not limited to, antagonist antibodies, polypeptide fragments, oligopeptides, organic molecules (including small molecules), and anti-sense nucleic acids.

The term “agonist” is used in the broadest sense, and includes any molecule that partially or fully mimics a biological activity of a polypeptide, or that increases the transcription or translation of a nucleic acid encoding the polypeptide. Suitable agonist molecules include, but are not limited to, agonist antibodies, polypeptide fragments, oligopeptides, organic molecules (including small molecules), polynucleotides, polypeptides, and polypeptide-Fc fusions.

The term “cytotoxic agent” as used herein refers to a substance that inhibits or prevents a cellular function and/or causes cell death or destruction. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu), chemotherapeutic agents (e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A “tumoricidal” agent causes destruction of tumor cells.

A “toxin” is any substance capable of having a detrimental effect on the growth or proliferation of a cell.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegall (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhône-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

“Antibodies” (Abs) and “immunoglobulins” (Igs) refer to glycoproteins having similar structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The term “anti-CD117 antibody” or “an antibody that binds to CD117” refers to an antibody that is capable of binding CD117 with sufficient affinity such that the antibody is useful as a diagnostic and/or therapeutic agent in targeting CD117. In certain embodiments, an antibody that binds to CD117 has a dissociation constant (Kd) of ≦1 μM, ≦100 nM, ≦10 nM, ≦1 nM, or ≦0.1 nM. In certain embodiments, an anti-CD117 antibody binds to an epitope of CD117 that is conserved among CD117 from different species.

The term “anti-CD44 antibody” refers to an antibody that is capable of binding CD44 with sufficient affinity such that the antibody is useful as a diagnostic and/or therapeutic agent in targeting CD44. In certain embodiments, an antibody that binds to CD44 has a dissociation constant (Kd) of ≦1 μM, ≦100 nM, ≦10 nM, ≦1 nM, or ≦0.1 nM. In certain embodiments, an anti-CD44 antibody binds to an epitope of CD44 that is conserved among CD44 from different species.

The term “anti-CD133 antibody” refers to an antibody that is capable of binding CD133 with sufficient affinity such that the antibody is useful as a diagnostic and/or therapeutic agent in targeting CD133. In certain embodiments, an antibody that binds to CD133 has a dissociation constant (Kd) of ≦1 μM, ≦100 nM, ≦10 nM, ≦1 nM, or ≦0.1 nM. In certain embodiments, an anti-CD133 antibody binds to an epitope of CD133 that is conserved among CD133 from different species.

The term “anti-Sca-1 antibody” refers to an antibody that is capable of binding Sca-1 with sufficient affinity such that the antibody is useful as a diagnostic and/or therapeutic agent in targeting Sca-1. In certain embodiments, an antibody that binds to Sca-1 has a dissociation constant (Kd) of ≦1 μM, ≦100 nM, ≦10 nM, ≦1 nM, or ≦0.1 nM. In certain embodiments, an anti-Sca-1 antibody binds to an epitope of Sca-1 that is conserved among Sca-1 from different species.

The terms “full length antibody,” “intact antibody” and “whole antibody” are used herein interchangeably to refer to an antibody in its substantially intact form, not antibody fragments as defined below. The terms particularly refer to an antibody with heavy chains that contain the Fc region.

“Antibody fragments” comprise only a portion of an intact antibody, wherein the portion retains at least one, and as many as most or all, of the functions normally associated with that portion when present in an intact antibody. In one embodiment, an antibody fragment comprises an antigen binding site of the intact antibody and thus retains the ability to bind antigen. In another embodiment, an antibody fragment, for example, one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise an antigen binding arm linked to an Fc sequence capable of conferring in vivo stability to the fragment.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is a minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO93/1161; Hudson et al. (2003) Nat. Med. 9:129-134; and Hollinger et al., Proc. Natl. Acad. Sci. USA 90: 6444-6448 (1993). Triabodies and tetrabodies are also described in Hudson et al. (2003) Nat. Med. 9:129-134.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256: 495 (1975); Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., Bio. Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996) and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which comprises an amino acid sequence corresponding to that of an antibody produced by a human and/or has been made using any of the techniques for making human antibodies as disclosed herein. Such techniques include screening human-derived combinatorial libraries, such as phage display libraries (see, e.g., Marks et al., J. Mol. Biol., 222: 581-597 (1991) and Hoogenboom et al., Nucl. Acids Res., 19: 4133-4137 (1991)); using human myeloma and mouse-human heteromyeloma cell lines for the production of human monoclonal antibodies (see, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)); and generating monoclonal antibodies in transgenic animals (e.g., mice) that are capable of producing a full repertoire of human antibodies in the absence of endogenous immunoglobulin production (see, e.g., Jakobovits et al., Proc. Natl. Acad. Sci. USA, 90: 2551 (1993); Jakobovits et al., Nature, 362: 255 (1993); Bruggermann et al., Year in Immunol., 7: 33 (1993)). This definition of a human antibody specifically excludes a humanized antibody comprising antigen-binding residues from a non-human animal.

An “affinity matured” antibody is one with one or more alterations in one or more CDRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of HVR and/or framework residues is described by: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

A “blocking antibody” or an “antagonist antibody” is one which inhibits or reduces a biological activity of the antigen it binds. Certain blocking antibodies or antagonist antibodies partially or completely inhibit the biological activity of the antigen.

A “small molecule” or “small organic molecule” is defined herein as an organic molecule having a molecular weight below about 500 Daltons.

An “CD117-binding oligopeptide” or an “oligopeptide that binds CD117” is an oligopeptide that is capable of binding CD117 with sufficient affinity such that the oligopeptide is useful as a diagnostic and/or therapeutic agent in targeting CD117. In certain embodiments, the extent of binding of a CD117-binding oligopeptide to an unrelated, non-CD117 protein is less than about 10% of the binding of the CD117-binding oligopeptide to CD117 as measured, e.g., by a surface plasmon resonance assay. In certain embodiments, a CD117-binding oligopeptide has a dissociation constant (Kd) of ≦1 μM, ≦100 nM, ≦10 nM, ≦1 nM, or ≦0.1 nM.

A “CD117-binding organic molecule” or “an organic molecule that binds CD117” is an organic molecule other than an oligopeptide or antibody as defined herein that is capable of binding CD117 with sufficient affinity such that the organic molecule is useful as a diagnostic and/or therapeutic agent in targeting CD117. In certain embodiments, the extent of binding of a CD117-binding organic molecule to an unrelated, non-CD117 protein is less than about 10% of the binding of the CD117-binding organic molecule to CD117 as measured, e.g., by a surface plasmon resonance assay. In certain embodiments, a CD117-binding organic molecule has a dissociation constant (Kd) of ≦1 μM, ≦100 nM, ≦10 nM, ≦1 nM, or ≦0.1 nM.

The dissociation constant (Kd) of any molecule that binds a target polypeptide may conveniently be measured using a surface plasmon resonance assay. Such assays may employ a BIAcore™-2000 or a BIAcore™-3000 (BIAcore, Inc., Piscataway, N.J.) at 25° C. with immobilized target polypeptide CMS chips at ˜10 response units (RU). Briefly, carboxymethylated dextran biosensor chips (CMS, BIAcore Inc.) are activated with N-ethyl-N′-(3-dimethylaminopropyl)-carbodiimide hydrochloride (EDC) and N-hydroxysuccinimide (NHS) according to the supplier's instructions. Target polypeptide is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of target polypeptide, 1 M ethanolamine is injected to block unreacted groups. For kinetics measurements, two-fold serial dilutions of the binding molecule (0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneously fitting the association and dissociation sensorgrams. The equilibrium dissociation constant (Kd) is calculated as the ratio k_{off}/kₒₙ. See, e.g., Chen, Y., et al., (1999) J. Mol. Biol. 293:865-881. If the on-rate of an antibody exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stirred cuvette.

A “liposome” is a small vesicle composed of various types of lipids, phospholipids and/or surfactant which is useful for delivery of an agent, e.g., a drug, to a mammal. The components of the liposome are commonly arranged in a bilayer formation, similar to the lipid arrangement of biological membranes.

The word “label” when used herein refers to a detectable compound or composition. The label may be detectable by itself (e.g., radioisotope labels or fluorescent labels) or, in the case of an enzymatic label, may catalyze chemical alteration of a substrate compound or composition which results in a detectable product. Radionuclides that can serve as detectable labels include, for example, I-131, I-123, I-125, Y-90, Re-188, Re-186, At-211, Cu-67, Bi-212, and Pd-109.

An “isolated” biological molecule, such as a nucleic acid, polypeptide, or antibody, is one which has been identified and separated and/or recovered from at least one component of its natural environment.

An “isolated cell” is a cell which has been identified and separated and/or recovered from at least one component of its natural environment.

### Compositions and Methods of the Invention

As provided herein, CD117 is a rare marker of prostate stem cells (PSCs) that possess multi-potent, self-renewing capacity. CD117 expression is predominantly localized to the region of the mouse prostate proximal to the urethra and is upregulated following castration-induced prostate involution, two characteristics consistent with that of a PSC marker. CD117⁺PSCs can generate functional, secretion-producing prostates when transplanted in vivo. Moreover, CD117⁺PSCs exhibit long-term self-renewal capacity, as evidenced by serial isolation and transplantation in vivo. Further purification of the PSCs is desired in some embodiments and is achieved by sorting or isolating cells based on the additional markers CD133, and/or CD44, and/or Sca-1. As described in the Examples, a single cell isolated from an adult mouse prostate defined by Lin-Sca-1⁺CD133⁺CD44⁺CD117⁺phenotype can generate a prostate upon transplantation in vivo.

Accordingly, a method of isolating PSCs is provided in one aspect of the invention. In one embodiment, a population of prostate cells is obtained from the prostate of a donor. In some embodiments, the donor is a mammalian donor. In some embodiments, the donor is a human. In other embodiments, the donor is a mouse, a rat, a pig, or other suitable mammalian donor. In some embodiments, the prostate cell population is treated to remove all lineage cells resulting in a lineage depleted (Lin−) cell population. Methods for obtaining a Lin− cell population are well known in the art and are described in the Examples. The prostate cell population is sorted to obtain a population of cells that expresses at least one, at least two, at least three, or at least four of the cell surface markers CD117, CD133, CD44, and Sca-1. In specific embodiments, the Lin− cell population is sorted to obtain a population of cells that express CD117, CD133, and CD44 (phenotype Lin−/CD117+/CD133+/CD44+) or CD117, CD133, CD44, Sca-1 (phenotype Lin−/CD117+/CD133+/CD44+/Sca-1+). Additional embodiments include, for example, methods of sorting cell populations to obtain cells with the following phenotypes, Lin−/CD117+, Lin−/CD117+/CD133+, Lin−/CD117+/CD44+, Lin−/CD117+/Sca-1+, Lin−/CD117+/CD133+/Sca-1+, Lin−/CD117+/CD44+/Sca-1+.

Methods of sorting cells based on cell surface markers are well known in the art and include, for example, standard flow cytometry, magnetic cell sorting, and fluorescence-activated cell sorting (FACS).

Another aspect of the invention provides for the cell population and/or single cells obtained from the cell sorting methods. The cell populations are substantially pure and do not contain a significant population of cells that do not express the markers used to sort the cells. In some embodiments, the cell population is at least 90%, 91%, 92%, 93%, 94%, 95%, 96%, 97%, 98%, 99%, or 99.5% pure.

PSCs obtained using the sorting methods can be used in a number of methods, including methods of prostate tissue regeneration and methods of screening for compounds.

The loss of a functional prostate in a human male often results in a number of undesirable physical and emotional conditions including loss of sexual function, urinary incontinence, bowel dysfunction, and depression. Kirby, R. S. et al., Prost Can Prost Disease 1:179-184 (1998), Weber, B. et al, Am J Men's Health, 2 (2): 165-171 (2008). Accordingly, one aspect of the invention provides for methods of regenerating a functioning prostate in a patient. In one embodiment, a PSC of the invention is implanted in a patient so that the PSC regenerates a functioning prostate. In one embodiment, the patient has no prostate and the PSC is implanted near the urethra of a patient resulting in generation of a prostate. In another embodiment, the patient has a partial prostate and the PSC is implant in or near the partial prostate resulting in regeneration of prostate tissue and a functioning prostate. In one embodiment, the patient is a human patient. In another embodiment, the patient is a human male patient.

In yet another embodiment, a host organism is used to generate a prostate for transplantation into a patient. In one embodiment, the patient is a human patient. In another embodiment, the patient is a human male patient. In one embodiment, a PSC from a patient, or from a member of the same species as the patient, is implanted in a host organism resulting in a generation of a prostate. The PSC is implanted in the host organism in a manner to provide sufficient vascularization to generate the prostate. In one embodiment, the PSC is implanted under the renal capsule of the host. The prostate is removed from the host and transplanted into a patient in need of a functioning prostate. In one embodiment, the host and patient are the same or from the same species. In one embodiment, the host and the patient are human. In another embodiment, the host and patient are not of the same species. In one embodiment, the host is a pig or other suitable non-human mammal. In another embodiment, the patient is a human.

In yet another embodiment, the prostate is generated in an ex vivo system. Systems for generating tissue and organs ex vivo are known and are described in, for example, U.S. Pat. Nos. 6,121,042, 6,210,957, 6,171,812, 7,410,792, 6,432,713, 6,599,734, 6,607,917, and 6,921,662.

The markers used to isolate normal PSCs are also useful in isolating prostate cancer stem cells (PCSCs). The PCSCs of the invention express the same markers used to isolate normal PSCs. The normal PSCs and PCSCs can be differentiated from one and other using assays that determine if the cells differentiate into prostates (normal PSCs) or proliferate and give rise to prostate cancer (PCSCs). Such assays are known in the art and described herein.

Both PSCs and PCSCs are useful in methods of screening for compounds that can be used to either increase tissue generation or to decrease cellular proliferation and to treat prostate cancer. Accordingly, one aspect of the invention provides for a method of identifying a compound for the treatment of prostate cancer. In one embodiment, the method comprises contacting a PSC or PCSC (or substantially pure population of the PSCs or PCSCs) with a test compound, and assessing the effect of the test compound on the proliferation or viability of the PSC or PCSC. In a particular embodiment, the PSC or PCSC expresses CD117. In another embodiment, the PSC or PCSC expresses CD117 and CD44. In another embodiment, the PSC or PCSC expresses CD117 and CD133. In another embodiment, the PSC or PCSC expresses CD117, CD44, and CD133. In another embodiment, the PSC or PCSC expresses CD117, CD44, CD133, and Sca-1. In one embodiment, the method determines if the test compound inhibits proliferation of the PSC or PCSC Inhibition of proliferation can be determined using any method known in the art including in vitro cellular proliferation assays. In some embodiments, the method includes determining proliferation of the PSC or PCSC in absence of the test compound to provide a comparison for the effect of the test compound. In another embodiment, the method determines if the test compound kills the PSC or PCSC.

Assays for inhibition of cell growth or proliferation are well known in the art. Certain assays for cell proliferation, exemplified by the “cell killing” assays described herein, measure cell viability. One such assay is the CellTiter-Glo™ Luminescent Cell Viability Assay, which is commercially available from Promega (Madison, Wis.). That assay determines the number of viable cells in culture based on quantitation of ATP present, which is an indication of metabolically active cells. See Crouch et al (1993) J. Immunol. Meth. 160:81-88, U.S. Pat. No. 6,602,677. The assay may be conducted in 96- or 384-well format, making it amenable to automated high-throughput screening (HTS). See Cree et al (1995) AntiCancer Drugs 6:398-404. The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU).

Another assay for cell proliferation is the “MTT” assay, a colorimetric assay that measures the oxidation of 3-(4,5-dimethylthiazol-2-yl)-2,5-diphenyltetrazolium bromide to formazan by mitochondrial reductase. Like the CellTiter-Glo™ assay, this assay indicates the number of metabolically active cells present in a cell culture. See, e.g., Mosmann (1983) J. Immunol. Meth. 65:55-63, and Zhang et al. (2005) Cancer Res. 65:3877-3882.

Assays for induction of cell death are well known in the art. In some embodiments, such assays measure, e.g., loss of membrane integrity as indicated by uptake of propidium iodide (PI), trypan blue (see Moore et al. (1995) Cytotechnology, 17:1-11), or 7AAD. In an exemplary PI uptake assay, cells are cultured in Dulbecco's Modified Eagle Medium (D-MEM):Ham's F-12 (50:50) supplemented with 10% heat-inactivated FBS (Hyclone) and 2 mM L-glutamine. Thus, the assay is performed in the absence of complement and immune effector cells. Cells are seeded at a density of 3×10⁶ per dish in 100×20 mm dishes and allowed to attach overnight. The medium is removed and replaced with fresh medium alone or medium containing various concentrations of the antibody or immunoconjugate. The cells are incubated for a 3-day time period. Following treatment, monolayers are washed with PBS and detached by trypsinization. Cells are then centrifuged at 1200 rpm for 5 minutes at 4° C., the pellet resuspended in 3 ml cold Ca²⁺ binding buffer (10 mM Hepes, pH 7.4, 140 mM NaCl, 2.5 mM CaCl₂) and aliquoted into 35 mm strainer-capped 12×75 mm tubes (1 ml per tube, 3 tubes per treatment group) for removal of cell clumps. Tubes then receive PI (10 μg/ml). Samples are analyzed using a FACSCAN™ flow cytometer and FACSCONVERT™ CellQuest software (Becton Dickinson). Compounds which induce statistically significant levels of cell death as determined by PI uptake are thus identified.

Exemplary assays for compounds that induce apoptosis is an annexin binding assays and histone DNA ELISA colorimetric assay for detecting internucleosomal degradation of genomic DNAs. Such an assay can be performed using, e.g., the Cell Death Detection ELISA kit (Roche, Palo Alto, Calif.).

Another aspect of the invention provides for a method of identifying a compound for promoting tissue regeneration. In one embodiment, the method comprises contacting a PSC (or substantially pure population of PSCs) with a test compound, and assessing the effect of the test compound on the growth and differentiation of the PSCs into prostate tissue, prostate colonies, or a functioning prostate. In a particular embodiment, the PSC expresses CD117. In another embodiment, the PSC expresses CD117 and CD44. In another embodiment, the PSC expresses CD117 and CD133. In another embodiment, the PSC expresses CD117, CD44, and CD133. In another embodiment, the PSC expresses CD117, CD44, CD133, and Sca-1. In some embodiments, the method includes determining the effect of the test compound on the growth and differentiation of the PSCs in absence of the test compound to provide a comparison for the effect of the test compound. Assays for determining the growth promoting effect of a compound are found in the examples.

In another aspect, a method of inhibiting the proliferation of a PSC or PCSC is provided, the method comprising exposing or contacting the PSC or PCSC to an antagonist of CD117. In a particular embodiment, the PSC or PCSC expresses CD117. In another embodiment, the PSC or PCSC expresses CD117 and CD44. In another embodiment, the PSC or PCSC expresses CD117 and CD133. In another embodiment, the PSC or PCSC expresses CD117, CD44, and CD133. In another embodiment, the PSC or PCSC expresses CD117, CD44, CD133, and Sca-1.

Another aspect of the invention provides for methods of treating prostate cancer based on the presence, or absence, of the PSCs or PCSCs in the prostate cancer of a patient. Patients whose prostate cancer contains PSCs or PCSCs are predicted to respond to treatment with a compound that would inhibit proliferation of PSCs or PCSCs. Thus, the presence or absence of the PSCs or PCSCs can be used to determine if a compound that inhibits proliferation of PSCs or PSCSs should be included in a patient's treatment regime. The treatment regime can be the primary treatment regime. The presence of PSCs or PSCSs is particularly useful in predicting whether the prostate cancer is likely to reoccur in a patient who has had an apparently successful primary treatment regime. If the presence of PSCs or PSCSs is detected in the prostate cancer then it is predicted that the patient is likely to experience a reoccurrence of the prostate cancer and is a candidate for adjuvant therapy with compound that inhibits proliferation of PSCSs, or otherwise prevents the PCSCs from generating prostate cancer.

One specific embodiment provides for a method of treating prostate cancer in a patient comprising determining if the patient's prostate cancer comprises a PSC or PCSC and administering to the patient a therapeutically effective amount of a CD117 antagonist if the patient's prostate cancer comprises a PSC or PCSC. Another embodiment provides for a method of treating prostate cancer in a patient comprising determining if the patient's prostate cancer comprises a PSC or PCSC and administering to the patient a therapeutically effective amount of a CD133 antagonist if the patient's prostate cancer comprises a PSC or PCSC. Another embodiment provides for a method of treating prostate cancer in a patient comprising determining if the patient's prostate cancer comprises a PSC or PCSC and administering to the patient a therapeutically effective amount of a CD44 antagonist if the patient's prostate cancer comprises a PSC or PCSC. Yet another embodiment provides for a method of treating prostate cancer in a patient comprising determining if the patient's prostate cancer comprises a PSC or PCSC and administering to the patient a therapeutically effective amount of a Sca-1 antagonist if the patient's prostate cancer comprises a PSC or PCSC. Another embodiment, provides for a method of treating prostate cancer in a patient comprising determining if the patient's prostate cancer comprises a PSC or PCSC and administering to the patient a therapeutically effective amount of a combination of a CD117 antagonist and at least one CD133 antagonist, CD44 antagonist, and/or Sca-1 antagonist if the patient's prostate cancer comprises a PSC or PCSC.

Another aspect of the invention relates to predicting whether a prostate cancer patient is likely to experience a reoccurrence of prostate cancer after completion of the primary therapy used to treat the prostate cancer. One embodiment provides for a method of predicting whether a prostate cancer patient is likely to experience a reoccurrence of prostate cancer comprising determining if the patient's prostate cancer comprises a PSC or PCSC and predicting that the patient is likely to experience a reoccurrence if the patient's prostate cancer comprises a PSC or PCSC. This prediction can be used to guide the further treatment of the patient and can lead to the use of adjuvant therapy. In one embodiment, the prediction that the patient is likely to experience a reoccurrence of prostate cancer is followed by adjuvant therapy comprising administering to the patient a CD117 antagonist. In another embodiment, the prediction that the patient is likely to experience a reoccurrence of prostate cancer is followed by adjuvant therapy comprising administering to the patient a CD133 antagonist. In another embodiment, the prediction that the patient is likely to experience a reoccurrence of prostate cancer is followed by adjuvant therapy comprising administering to the patient a CD44 antagonist. In another embodiment, the prediction that the patient is likely to experience a reoccurrence of prostate cancer is followed by adjuvant therapy comprising administering to the patient a Sca-1 antagonist. In another embodiment, the prediction that the patient is likely to experience a reoccurrence of prostate cancer is followed by adjuvant therapy comprising administering to the patient a therapeutically effective amount of a combination of a CD117 antagonist and at least one CD133 antagonist, CD44 antagonist, and/or Sca-1 antagonist,

Another embodiment provides for a method of preventing reoccurrence of prostate cancer in a patient comprising determining if the patient's prostate cancer comprises a PSC or PCSC and administering to the patient a therapeutically effective amount of a CD117 antagonist if the patient's prostate cancer comprises a PSC or PCSC. A further embodiment provides for a method of providing adjuvant therapy to a patient treated for prostate cancer comprising determining if the patient's prostate cancer comprises a PSC or PCSC and administering to the patient a therapeutically effective amount of a CD117 antagonist if the patient's prostate cancer comprises a PSC or PCSC. Another embodiment provides for a method of providing adjuvant therapy to a patient treated for prostate cancer comprising determining if the patient's prostate cancer comprises a PSC or PCSC and administering to the patient a therapeutically effective amount of a CD133 antagonist if the patient's prostate cancer comprises a PSC or PCSC. Another embodiment provides for a method of providing adjuvant therapy to a patient treated for prostate cancer comprising determining if the patient's prostate cancer comprises PSC or PCSC and administering to the patient a therapeutically effective amount of a CD44 antagonist if the patient's prostate cancer comprises a PSC or PCSC. Another embodiment provides for a method of providing adjuvant therapy to a patient treated for prostate cancer comprising determining if the patient's prostate cancer comprises a PSC or PCSC and administering to the patient a therapeutically effective amount of a Sca-1 antagonist if the patient's prostate cancer comprises a PSC or PCSC. Another embodiment provides for a method of providing adjuvant therapy to a patient treated for prostate cancer comprising determining if the patient's prostate cancer comprises a PSC or PCSC and administering to the patient a therapeutically effective amount of a combination of a CD117 antagonist and at least one CD133 antagonist, CD44 antagonist, and/or Sca-1 antagonist,

Another embodiment provides a method of selecting a prostate cancer patient for treatment with a CD117 antagonist comprising determining if the patient has a prostate cancer that comprises PSC or PCSC and selecting the patient for treatment with a CD117 antagonist if the patient has a prostate cancer that comprises a PSC or PCSC.

Yet another embodiment provides for a method of selecting a prostate cancer patient for adjuvant treatment with a CD117 antagonist comprising determining if the patient's prostate cancer comprises a PSC or PCSC and selecting the patient for adjuvant treatment with a CD117 antagonist if the patient's prostate cancer comprises a PSC or PCSC.

In specific embodiments of the above aspects, the PSC or PCSC expresses CD117. In another embodiment, the PSC or PCSC expresses CD117 and CD44. In another embodiment, the PSC or PCSC expresses CD117 and CD133. In another embodiment, the PSC or PCSC expresses CD117, CD44, and CD133. In another embodiment, the PSC or PCSC expresses CD117, CD133, CD44, and Sca-1.

Detection of the expression of the markers CD117, CD133, CD44, and Sca-1 can be performed by any method known in the art. In one embodiment, marker overexpression is detected by determining the level of mRNA transcription from the marker gene. Levels of mRNA transcription may be determined, either quantitatively or qualitatively, by various methods known to those skilled in the art. Levels of mRNA transcription may also be determined directly or indirectly by detecting levels of cDNA generated from the mRNA. Exemplary methods for determining levels of mRNA transcription include, but are not limited to, PCR, real-time quantitative RT-PCR and hybridization-based assays, including microarray-based assays and filter-based assays such as Northern blots.

In other embodiments, expression of the marker is detected by determining the level of marker polypeptide expression. Levels of marker polypeptide may be determined, either quantitatively or quantitatively, by certain methods known to those skilled in the art, including antibody-based detection methods. In one embodiment, detecting expression of the marker gene in a test sample comprises contacting the test sample with an antibody specific for the marker polypeptide and determining the level of expression (either quantitatively or qualitatively) of marker polypeptide in the test sample by detecting binding of the antibody to marker polypeptide. In certain embodiments, binding of an antibody to a marker polypeptide may be detected by various methods known to those skilled in the art including, but not limited to, immunohistochemistry, fluorescence activated cell sorting, Western blot, radioimmunoassay, ELISA, and the like.

A sample of the cancer cells, or test sample, preferably comprises cells taken directly from the prostate cancer tumor, but the test sample can also be comprised of metastatic cancer cells, circulating tumor cells, or any suitable sample of cells that identify the amplification or expression status of the marker genes or polypeptides in the cancer.

In some embodiments, a control can be generated by determining the expression of a housekeeping gene (such as an actin family member) in the same test sample used to determine marker expression, or in a sample from the same cancer to be tested for marker expression. The housekeeping gene acts as a comparative control on which to determine expression of the marker gene.

In specific embodiments of the above aspects, the antagonist of CD117 is a small molecule antagonist. In another embodiment, the antagonist of CD117 is an antagonist antibody. In another embodiment, the antagonist of CD117 is soluble CD117 receptor or variant thereof.

A variety of CD117 kinase antagonists are known in the art. Such kinase antagonists include, but are not limited to antagonist antibodies and small molecule antagonists, e.g., 3-[2,4-dimethylpyrrol-5-yl)methylidene]-indoLin-2-one (“SU5416”); 5-[1,2-Dihydro-2-oxo-3H-indol-3-ylidene)methyl]-2,4-dim ethyl-1H-pyrrole-3-propanoic acid (“SU6668”); imatinib mesylate (“STI571”, Gleevec®, Novartis), sunitinib malate (Sutent®, Pfizer), 3-Phenyl-1H-benzofuro[3,2-c]pyrazole (“GTP-14564”), 5-[(Z)-(5-Chloro-2-oxo-1,2-dihydro-3H-indol-3-ylidene)methyl]-N-[2-(diethylamino)ethyl]-2,4-dimethyl-1H-pyrrole-3-carboxamide (“SU11652”), 6,7-Dimethoxy-3-phenylquinoxaline (“AG 1296”), 1,2-Dimethyl-6-(2-thienyl)-imidazolo[5,4-g]quinoxaline (“AGL 2043”). and indolinones such as 3-[3-(2-carboxyethyl)-4-methylpyrrol-2-methylidenyl]-2-indolinone (“SU5402”) (see, e.g., Bernard-Pierrot (2004) Oncogene 23:9201-9211).

In specific embodiments of the above aspects, the antagonist of CD133, CD44, or Sca-1 is a small molecule antagonist. In another embodiment, the antagonist of CD133, CD44, or Sca-1 is an antagonist antibody. In another embodiment, the antagonist of CD133, CD44, or Sca-1 is a variant of CD133, CD44, or Sca-1, respectively.

### Pharmaceutical Compositions

Therapeutic formulations comprising the antagonists are included and are prepared using standard methods known in the art by mixing the active ingredient having the desired degree of purity with optional physiologically acceptable carriers, excipients or stabilizers (Remington's Pharmaceutical Sciences (20.sup.th edition), ed. A. Gennaro, 2000, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

An antagonist described herein, such as a CD117 antagonist, is administered to a human subject, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Local administration may be particularly desired if extensive side effects or toxicity is associated with the specific antagonism. An ex vivo strategy can also be used for therapeutic applications. Ex vivo strategies involve transfecting or transducing cells obtained from the subject with a polynucleotide encoding an antibody or antibody fragment. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells.

In one example, the therapeutic compound is administered locally, e.g., by direct injections, when the disorder or location of the tumor permits, and the injections can be repeated periodically. An antagonist can also be delivered systemically to the subject or directly to the tumor cells, e.g., to a tumor or a tumor bed following surgical excision of the tumor, in order to prevent or reduce local recurrence or metastasis.

For the prevention or treatment of disease, the appropriate dosage of an antagonist of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. An antibody is suitably administered to the patient at one time or over a series of treatments. Depending on the type and severity of the disease, about 1 μg/kg to 20 mg/kg (e.g. 0.1 mg/kg-15 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 20 mg/kg. Thus, one or more doses of about 0.5 mg/kg, 2.0 mg/kg, 4.0 mg/kg, 10 mg/kg, 15 mg/kg, or 20 mg/kg (or any combination thereof) may be administered to the patient. Such doses may be administered intermittently, e.g. every week, every two weeks, or every three weeks (e.g. such that the patient receives from about two to about twenty, or e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

### Combination Therapy

An antagonist of the invention may be combined in a pharmaceutical combination formulation, or dosing regimen as combination therapy, with a second compound having anti-cancer properties. The second compound of the pharmaceutical combination formulation or dosing regimen may have complementary activities to the antibody of the combination such that they do not adversely affect each other.

The second compound may be an antibody, a chemotherapeutic agent, cytotoxic agent, cytokine, growth inhibitory agent, anti-hormonal agent, and/or cardioprotectant. Such molecules are suitably present in combination in amounts that are effective for the purpose intended. A pharmaceutical composition containing a compound of the invention may also have a therapeutically effective amount of a chemotherapeutic agent such as a tubulin-forming inhibitor, a topoisomerase inhibitor, a DNA intercalator, or a DNA binder.

Other therapeutic regimens may be combined with the administration of an antagonist identified in accordance with this invention. The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein there is a time period while both (or all) active agents simultaneously exert their biological activities.

As discussed above, certain embodiments of the invention provide for combinations of a CD117 antagonist and a CD133 antagonist, CD44 antagonist, or Sca-1 antagonist. Further embodiments provide for combinations of a CD117 antagonist and more than one CD133 antagonist, CD44 antagonist, and/or Sca-1 antagonist.

Additional examples of combination therapy include combinations with chemotherapeutic agents such as erlotinib (TARCEVA®, Genentech/OSI Pharm.), bortezomib (VELCADE®, Millenium Pharm.), fulvestrant (FASLODEX®, AstraZeneca), sutent (SU11248, Pfizer), letrozole (FEMARA®, Novartis), PTK787/ZK 222584 (Novartis), oxaliplatin (Eloxatin®, Sanofi), 5-FU (5-fluorouracil), leucovorin, Rapamycin (Sirolimus, RAPAMUNE®, Wyeth), lapatinib (TYKERB®, GSK572016, GlaxoSmithKline), lonafarnib (SCH 66336), sorafenib (BAY43-9006, Bayer Labs.), and gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics, calicheamicin, calicheamicin gamma1I and calicheamicin omegaI1; dynemicin, including dynemicin A; bisphosphonates, such as clodronate; an esperamicin; as well as neocarzinostatin chromophore and related chromoprotein enediyne antibiotic chromophores, aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.), ABRAXANE™ Cremophor-free, albumin, nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINEO vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Such combination therapy also includes: (i) anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON• toremifene; (ii) aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in abherant cell proliferation, such as, for example, PKC-alpha, Ralf and H-Ras; (viii) ribozymes such as a VEGF expression inhibitor (e.g., ANGIOZYME® ribozyme) and a HER2 expression inhibitor; (ix) vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; (x) anti-angiogenic agents such as bevacizumab (AVASTIN®, Genentech); and (xi) pharmaceutically acceptable salts, acids or derivatives of any of the above.

Preparation and dosing schedules for such chemotherapeutic agents may be used according to manufacturer's instructions or as determined empirically by the skilled practitioner. Preparation and dosing schedules for such chemotherapy are also described in Chemotherapy Service, (1992) Ed., M. C. Perry, Williams & Wilkins, Baltimore, Md.

The combination therapy may provide “synergy” and prove “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, i.e. serially, whereas in combination therapy, effective dosages of two or more active ingredients are administered together.

### Articles of Manufacture and Kits

Another embodiment of the invention is an article of manufacture containing materials useful for the treatment of cancers. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is a multispecific antibody or antibody fragment antibody of the invention. The label or package insert indicates that the composition is used for treating the particular condition. The label or package insert will further comprise instructions for administering the composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In one embodiment, the package insert indicates that the composition is used for treating prostate cancer. In another embodiment, the package insert indicates that the composition is used for treating prostate cancer's that comprise a PSC or PCSC as described herein.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The label or package insert may provide a description of the composition as well as instructions for the intended in vitro or diagnostic use.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

Commercially available reagents referred to in the Examples were used according to manufacturer's instructions unless otherwise indicated. The source of those cells identified in the following Examples, and throughout the specification, by ATCC accession numbers is the American Type Culture Collection, Manassas, Va. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

### EXAMPLES

### Example 1

### Methods

Animals.

Pregnant SD rats and C57BL/6 male mice (postnatal day 4 and 8-10 week old) were purchased from Charles River Laboratories, athymic nu/nu male mice (6-8 week old) were purchased from Harlan Sprague Dawley, and WBB6F1/J male mice (wildtype or W/W^{v}; 4-8 week old) were purchased from The Jackson Laboratory. The W allele encodes a CD117 gene with a deletion of the transmembrane domain and the amino terminus of the kinase domain, whereas the W^{v} allele encodes a CD117 gene with a single point mutation.

Antibodies.

Antibodies were purchased from the following sources—BD Biosciences: APC-conjugated CD117 (anti-mouse: clone 2B8; anti-human: clone YB5.B8), PE-Cy7-conjugated Sca-1 (clone D7), Ki67 (clone B56), E-cadherin (clone 36), active caspase3 (polyclonal 557035); eBioscience: PE-conjugated CD133 (anti-mouse: clone 13A4), APC-Alexa Fluor® 750-conjugated CD44 (anti-mouse/human: clone IM7), function-blocking CD117 (clone ACK2); Miltenyi Biotec: PE-conjugated CD133 (anti-human: clone AC133); Abcam: CK18 (clone C-04), H-2k^{b} (clone ER-HR52), CD117 (polyclonal ab956); Chemicon: mouse-specific β1 integrin (clone MB1.2), synaptophysin (clone SY38); R&D Systems: CD117 (clone 180627); Covance: CK14 (polyclonal AF64); AbD Serotec: CD31 (clone 2H8); Sigma: α-SMA (clone 1A4); Santa Cruz Biotechnology: probasin (polyclonal M-18), p63 (clone 4A4); Invitrogen: synaptophysin (polyclonal Z66), secondary antibodies conjugated to Alexa Fluor® 488 or 594. Nkx3.1 polyclonal antibody was a gift of C. Abate-Shen (UMDNJ-Robert Wood Johnson Medical School, New Jersey).

UGM Stromal Cell Preparation.

The urogenital sinus mesenchyme (UGM) isolation procedure has been described previously¹⁸. Briefly, E18 embryos from pregnant SD rats were sacrificed, and urogenital sinuses harvested. Following separation of the UGM from the urogenital sinus epithelium, the UGM was digested with 1 mg ml⁻¹ collagenase/dispase (Roche) in DMEM supplemented with 10% fetal bovine serum, 2 mM glutamine, 100 U ml⁻¹ penicillin, and 100 mg ml⁻¹ streptomycin for 60 min at 37° C., washed twice in prostate culture medium (DMEM supplemented with 10% fetal bovine serum, 2 mM glutamine, 10 μg mL⁻¹ insulin, 5.5 μg mL⁻¹ transferrin, 6.7 ng mL⁻¹ selenium, 1 nM testosterone (Innovative Research of America), 100 U ml⁻¹ penicillin, and 100 mg ml⁻¹ streptomycin), and cultured in the same medium in 24 well plates coated with 10 μg ml⁻¹ collagen type I. UGM cells were passaged at confluency by trypsin digestion and cultured in vitro for up to 1 week.

Prostate Cell Preparation.

Freshly resected human prostate specimens (both benign prostatic hyperplasia (BPH) and benign non-BPH specimens, distinguished via gross examination by a pathologist; wet weights between 1-3 g) were obtained from Bio-options Inc. and The University of California, San Francisco, from consenting patients in accordance with federal and state guidelines. Human and mouse prostates were minced, placed in DMEM supplemented with 10% fetal bovine serum, 2 mM glutamine, 100 U ml⁻¹ penicillin, and 100 mg ml⁻¹ streptomycin, digested with 1 mg ml⁻¹ collagenase/dispase for 90 min at 37° C. with agitation, and passed through a 70 μm filter.

Serial Isolation/Transplantation In Vivo.

For secondary transplants of CD117⁺ cells, primary grafts were magnetically-sorted (˜49,000 dissociated cells were obtained per primary graft, with CD117⁺ cells constituting 19% of magnetically-sorted cells), and sorted cells (10,000 cells per graft) were mixed with UGM stromal cells (250,000 cells per graft). For tertiary transplants of CD117⁺ cells, secondary grafts were magnetically-sorted (˜40,000 dissociated cells were obtained per secondary graft, with CD117⁺ cells constituting 11% of magnetically-sorted cells), and sorted cells (2,200 cells per graft) were mixed with UGM stromal cells (250,000 cells per graft). For secondary transplants of Lin⁻Sca-1⁺CD133⁺CD44⁺CD117⁺ cells, primary grafts were sorted by FACS (˜31,000 dissociated cells were obtained per secondary graft, with Lin⁻Sca-1⁺CD133⁺CD44⁺CD117⁺ cells constituting 0.02% of viable FACS-sorted cells). Sorted cells (15 cells per graft) were mixed with UGM stromal cells (250,000 cells per graft). All serial transplantation grafts were harvested 12 weeks post-implantation. Gross graft images were acquired on a SMZ 800 dissecting microscope (Nikon) with a Coolpix® 4300 digital camera (Nikon).

RNA Isolation and Q-RT-PCR.

Prostates from 8-10 week old C57BL/6 mice were harvested and teased apart to extend the tubules. For comparison of prostatic regions, each prostatic lobe was divided into distal/intermediate/proximal regions. For comparison of prostatic lobes, each prostate was divided into dorsal/lateral/ventral/anterior lobes. Total RNA was isolated using an RNeasy® Mini kit (Qiagen) and Q-RT-PCR was performed with Power SYBR® Green (Applied Biosystems) using the following primer sets: Sca-1,5′-ATGGACACTTCTCACACTACAAAG-3′ (SEQ ID NO: 1) and 5′-TCAGAGCAAGGTCTGCAGGAGGACTG-3′ (SEQ ID NO: 2); CD44, 5′-AATTCCGAGGATTCATCCCA-3′ (SEQ ID NO: 3) and 5′-CGCTGCTGACATCGTCATC-3 (SEQ ID NO: 4); CD49b, 5′-CCGGCATACGAAAGAATTGG-3′ (SEQ ID NO: 5) and 5′-GAAGAGCTGAGGGTTATGT-3′ (SEQ ID NO: 6); CD49f, 5′-GTGGCCCAAGGAGATTAGC-3′ (SEQ ID NO: 7) and 5′-GTTGACGCTGCAGTTGAGA-3′ (SEQ ID NO: 8); CD133, 5′-ACCAACACCAAGAACAAGGC-3′ (SEQ ID NO: 9) and 5′-GGAGCTGACTTGAATTGAGG-3 (SEQ ID NO: 10); Bcl2, 5′-ATGTGTGTGGAGAGCGTCAAC-3′ (SEQ ID NO: 11) and 5′-AGACAGCCAGGAGAAATCAAAC-3′ (SEQ ID NO: 12); TERT, 5′-ATGGCGTTCCTGAGTATG-3′ (SEQ ID NO: 13) and 5′-TTCAACCGCAAGACCGACAG-3′ (SEQ ID NO: 14); p63, 5′-TTGTACCTGGAAAACAATG-3′ (SEQ ID NO: 15) and 5′-TCGAAGCTGTGTGGGCCCGGG-3 (SEQ ID NO: 16); CK14, 5′-GACTTCCGGACCAAGTTTGA-3′ (SEQ ID NO: 17) and 5′-CTTGAGGCTCTCAATCTGC-3′ (SEQ ID NO: 18); CK18, 5′-ACTCCGCAAGGTGGTAGATG-3′ (SEQ ID NO: 19) and 5′-GCCTCGATTTCTGTCTCCAG-3′ (SEQ ID NO: 20); CD24, 5′-TAAAGGACGCGTGAAAGGTTTGA-3′ (SEQ ID NO: 21) and 5′-GACAAAATGGGTCTCCATTCCGCAC-3′ (SEQ ID NO: 22); CD34, 5′-ATGCAGGTCCACAGGGACACG-3′ (SEQ ID NO: 23) and 5′-CTGTCCTGATAGATCAAGTAG-3′ (SEQ ID NO: 24); CD117, 5′-GACGCAACTTCCTTATGATC-3′ (SEQ ID NO: 25) and 5′-TGGTTTGAGCATCTTCACGG-3′ (SEQ ID NO: 26); Slug, 5′-TTTCTCCAGACCCTGGCTGCT-3′ (SEQ ID NO: 27) and 5′-TTTTCCCCAGTGTGAGTTCTA-3′ (SEQ ID NO: 28); GAPDH, 5′-ACTGGCATGGCCTTCCG-3′ (SEQ ID NO: 29) and 5′-CAGGCGGCACGTCAGATC-3′ (SEQ ID NO: 30). Gene expression was normalized to GAPDH using the ΔCt method.

Flow Cytometry.

Prostate cells (non-lineage depleted) were permeabilized with 0.1% Triton® X-100, stained with primary (CK14, CK18, APC-conjugated CD117) and secondary (Alexa Fluor® 488 or 594) antibodies, and analyzed on a LSR-II flow cytometer (Becton Dickinson).

Colony Formation In Vitro.

Prostate cells from 8-10 week old C57BL/6 mice were magnetically-sorted into CD117^{+/-} fractions, resuspended at 8,000 cells per 100 ml collagen type I at 3 mg ml⁻¹ in DMEM, placed in flat-bottom 96 well plates for 1 h at 37° C., and overlaid with prostate culture medium supplemented with 15 ng ml⁻¹ epidermal growth factor (Roche). Medium was changed every 48 h, and colony formation was assessed after 7 days.

Prostate Culture Ex Vivo.

Postnatal day 4 C57BL/6 mouse prostates were harvested and placed on 8 μm pore-size cell culture inserts (BD Falcon®), and inserts were placed into 24 well plates containing 300 μl DMEM/F-12 supplemented with 0.5% glucose, 2 mM glutamine, 10 μg mL⁻¹ insulin, 5.5 μg mL⁻¹ transferrin, 6.7 ng mL⁻¹ selenium, 100 U ml⁻¹ penicillin, 100 mg ml⁻¹ streptomycin, and 25 ng ml⁻¹ fungizone. Medium supplemented with function-blocking anti-CD117 antibody (25 μg ml⁻¹) was also used.

Medium was changed and images were acquired every 48 h, and prostates were harvested after 10 days. Images were acquired on a MZ16FA dissecting microscope (Leica) with a Retiga EXi digital camera (QImaging). Net growth in prostate area was quantified using MetaMorph® software (Molecular Devices). Branch point quantification was performed on gross images of day 8 prostates.

### Castration and Androgen Replacement.

For microarray and Q-RT-PCR analysis: C57BL/6 mice at 8-10 weeks of age were used. On day 0, mice were castrated. On days 3 and 14 post-castration, prostates from a subset or mice were harvested. On day 14 post-castration, testosterone pellets (15 mg/pellet/mouse) were implanted. On day 17 (3 days post-hormone replacement), prostates were harvested. Total RNA was isolated using an RNeasy® Mini kit, and MOE430v2 Affymetrix® chips were used for microarray analysis. For assessing CD117 function during prostate regeneration in vivo: C57BL/6 mice at 8 weeks of age were used. On day 0, mice were castrated. On day 12 post-castration, anti-ragweed control antibody (10 mg kg⁻¹ in PBS) or function-blocking anti-CD117 antibody (10 mg kg⁻¹ in PBS) were administered via i.p. injection. On day 14 post-castration, testosterone pellets (15 mg/pellet/mouse) were implanted. On day 15 (1 day post-hormone replacement), antibody treatments were administered. On day 19 (5 days post-hormone replacement), prostates were harvested, weighed, and processed for histology. Prostate weights are expressed as the net increase over control day 14 post-castration prostates.

Immunohistochemistry.

OCT-frozen tissues were sectioned at 8 μm, fixed in 4% paraformaldehyde (for CK14, CK18, CD117, synaptophysin, probasin, β1 integrin, H-2k^{b}, CD31, SMA) or methanol:acetone (1:1 vol/vol; for E-cadherin, activated caspase3, Ki67, Nkx3.1), and incubated with primary antibody for 45 min and secondary antibody for 30 min. Human prostate specimens were fixed in formalin and sectioned at 6 μm, and antigen retrieval was performed with BD Retrievagen A (BD Biosciences). For specificity controls, species-matched primary antibodies were used. Images were acquired on an Axioplan™ 2 imaging microscope (Zeiss) with an ORCA-ER digital camera (Hamamatsu). For ex vivo prostates, percentages of positive cells for CK14/CK18, Ki67, and E-cadherin were quantified by assessing at least 600, 400, and 1,200 cells, respectively. For regenerated in vivo prostates, percentages of positive cells for CK14/CK18 and Ki67 were quantified by assessing at least 800 and 2,500 cells, respectively.

Laser Capture Microdissection and PCR-Based Genotyping.

Single cell-derived grafts were sectioned at 8 μm, mounted on metal frame membrane slides (Molecular Machines & Industries), and stained with mouse-specific β1 integrin and CD31. Within the graft, β1 integrin⁺/CD31⁻ cells were isolated with a Nikon E2000 CellCut laser capture microdissector (Molecular Machines & Industries). For Foxn1^{+/+} cell controls, rat stromal cells (β1 integrin⁻/CD31⁻) within the graft were isolated. For Foxn1^{+/-} cell controls, athymic nu/nu kidney cells (β1 integrin⁺) adjacent to the graft were isolated. Captured cells were lysed with a PicoPure™ DNA Extraction Kit (Molecular Devices), and PCR-based genotyping was performed (http://jaxmice.jax.org/pub-cgi/protocols/protocols.sh). Genomic DNA was amplified by PCR with primers for Foxn1 (5′-GGCCCAGCAGGCAGCCCAAG-3′ (SEQ ID NO: 31) and 5′-AGGGATCTCCTCAAAGGCTTC-3′ (SEQ ID NO: 32)), digested with BsaJI, and run on a 4% agarose gel. Undigested PCR product is 168 bp; digested Foxn1^{+/+}PCR product gives 90 bp, 58 bp, and 20 bp fragments; digested Foxn1^{+/-} PCR product gives 110 bp, 90 bp, 58 bp, and 20 bp fragments. The absence of a 110 bp product indicates that genomic DNA is derived from Foxn1^{+/+} (wildtype) cells. PCR control reactions included water (negative control) and wildtype mouse genomic DNA (positive control).

Confocal and Single Cell Microscopy.

Confocal images were scanned and acquired with a LSM 510 META confocal microscope (Zeiss). Single cell images were acquired on an Eclipse TE300 inverted microscope (Nikon) with a Cascade Photometrics digital camera (Roper Scientific).

Limiting Dilution Analysis.

Limiting dilution analysis was performed using the ‘limdil’ function in the ‘statmod’ software package (http://bioinf.wehi.edu.au/software/limdil/index.html). A confidence interval of 95% was used.

Statistical Analysis.

Group differences were evaluated by two-tailed Student's t test. P values less than 0.05 were considered significant.

Prostate Generation In Vivo.

The prostate generation assay was described previouslyl^{7,18}. For primary transplants of CD117⁺ cells, prostates from 8-10 week old C57BL/6 mice were dissociated and magnetically-sorted with anti-mouse CD117 microbeads (Miltenyi Biotec) into CD117^{+/-} fractions (˜500,000 dissociated cells were obtained per prostate, with CD117⁺ cells constituting 7% of magnetically-sorted cells, of which 17.5±2.4% (n=10) were viable as determined by flow cytometry). Sorted cells (100,000 cells per graft) were mixed with UGM stromal cells (250,000 cells per graft) in 3 mg ml⁻¹ collagen type I (20 μl per graft), incubated at 37° C. for 1 h to allow collagen gelation, and overlaid with prostate culture medium. Following 37° C. overnight incubation, collagen gels were grafted under the renal capsule of 6-8 week old athymic nu/nu mice, along with a subcutaneous 90-day slow-release testosterone pellet (12.5 mg/pellet/mouse; Innovative Research of America). Grafts were harvested 8 weeks post-implantation. For primary transplants of Lin⁻Sca-1⁺CD133⁺CD44⁺CD117⁺ cells, prostates were sorted by FACS, and sorted cells (1,300 cells per graft) were mixed with UGM stromal cells (250,000 cells per graft). Grafts were harvested 12 weeks post-implantation.

### Single Cell FACS and Prostate Generation In Vivo.

Details of the procedure are described in Example 8. Prostates from 8-10-week-old C57BL/6 mice were dissociated and lineage-depleted using a Mouse Lineage Cell Depletion Kit (Miltenyi Biotec), along with a biotin-conjugated anti-mouse CD31 monoclonal antibody (BD Biosciences; clone 390). FACS was performed with a FACSAria flow cytometer (Becton Dickinson). Compensation adjustments were performed with single colour positive controls. Single cells were sorted into Microtest U-bottom 96 well plates (BD Falcon) containing 20 ml collagen type I at 3 mg ml 21. A total of 127 individual wells from single cell FACS were examined, with 106 wells verified to contain a single viable cell from six independent experiments.

### Example 2

### Identification of Prostate Stem Cell Markers

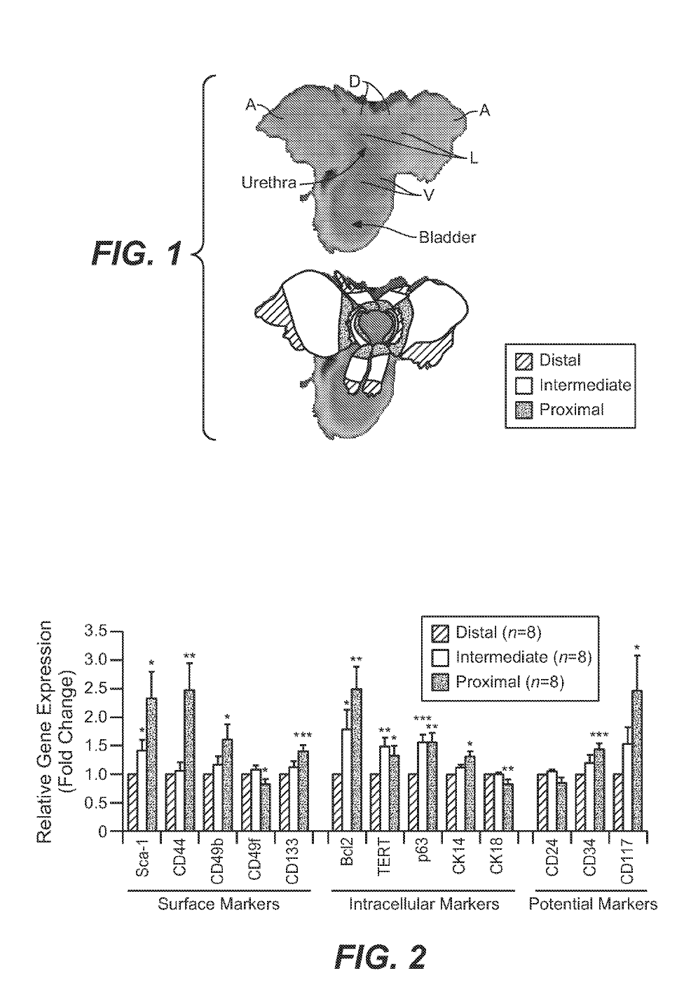

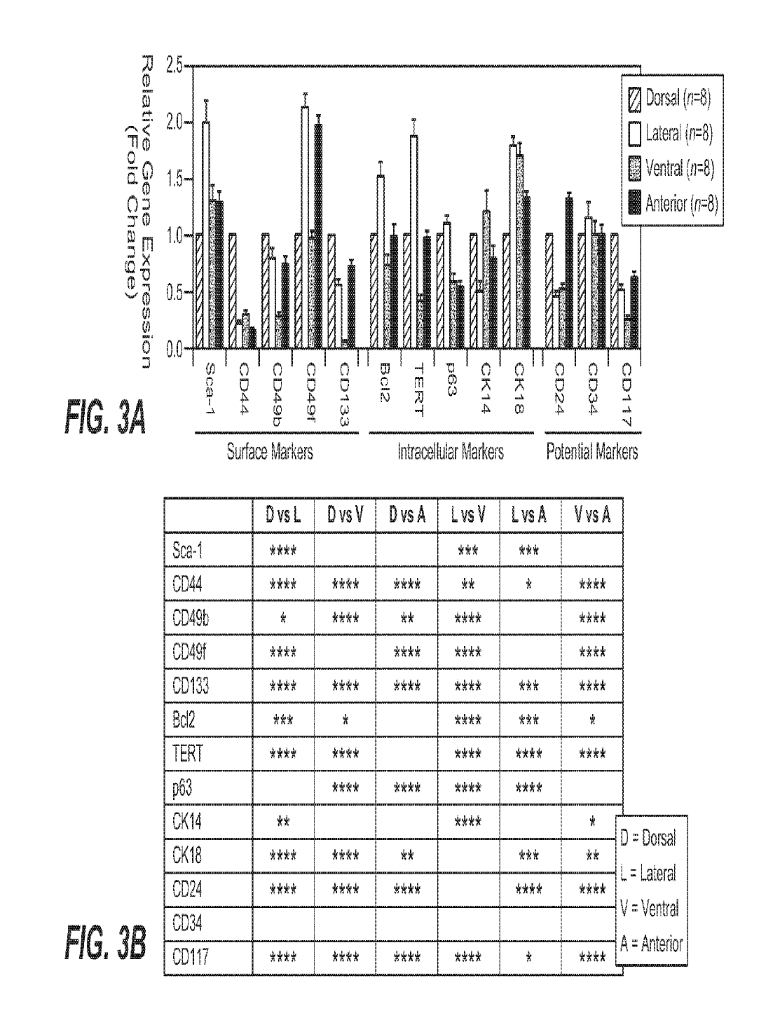

The mouse prostate is a branched ductal network consisting of four pairs of lobes (dorsal/lateral/ventral/anterior) with each lobe divided into three regions relative to the urethra (distal/intermediate/proximal; FIG. 1)¹¹. Wildtype prostates were dissected into distal/intermediate/proximal regions and quantitative reverse transcriptase polymerase chain reaction (Q-RT-PCR) was performed to identify stem cell markers.¹²⁻¹⁴. Four cell-surface markers (Sca-1, CD44, CD49b, and CD133), all known markers of PSCs^{2-6,8}, and three intracellular stem cell markers (Bcl2, telomerase reverse transcriptase (TERT), and p63), exhibited preferential expression in the proximal region (FIG. 2 and FIG. 3a/3b), thus confirming the validity of this assay system. The fact that CD44, CD49b, CD133, Bcl2, TERT, and p63 are all prostatic basal markers^{3-5,8,15,16} suggests that basal markers, relative to luminal markers, may be expressed at greater levels in the proximal region. Consistent with this, the basal marker cytokeratin 14 (CK14) exhibited preferential expression in the proximal region, with an opposite pattern observed for the luminal marker CK18. These data support that PSCs may constitute a subpopulation of basal cells, as previously proposed^{8,9}.

The expression level of stem cell-surface markers not previously reported to identify normal PSCs (CD24, CD34, and CD117) were assessed. CD34 and CD117, but not CD24, were predominantly expressed in the proximal region. Because CD117 exhibited greater differential expression between the proximal and distal regions compared to CD34, CD117 was focused on as a potential PSC marker Immunostaining confirmed a basal CD117⁺CK14⁺ population with a predominant proximal expression pattern. A CD117⁺CK14⁻ population, however, was also observed in the proximal region with subsequent analysis identifying a luminal CD117⁺CK18⁺ population. Flow cytometry was performed with triple labeling for CK14, CK18, and CD117. Although CD117⁺cells were enriched in the basal compartment, these findings indicated that CD117⁺ cells were not exclusively localized to either the basal or luminal compartments. Moreover, CD117 expression was not confined to a particular prostatic lobe. Rather, CD117, along with CD44, CD49b, and CD133, were expressed in all 4 pairs of lobes, with prominent expression detected in the dorsal prostate (FIGS. 3a and 3b). Hence CD117 exhibits an expression profile similar to that of known PSC markers.

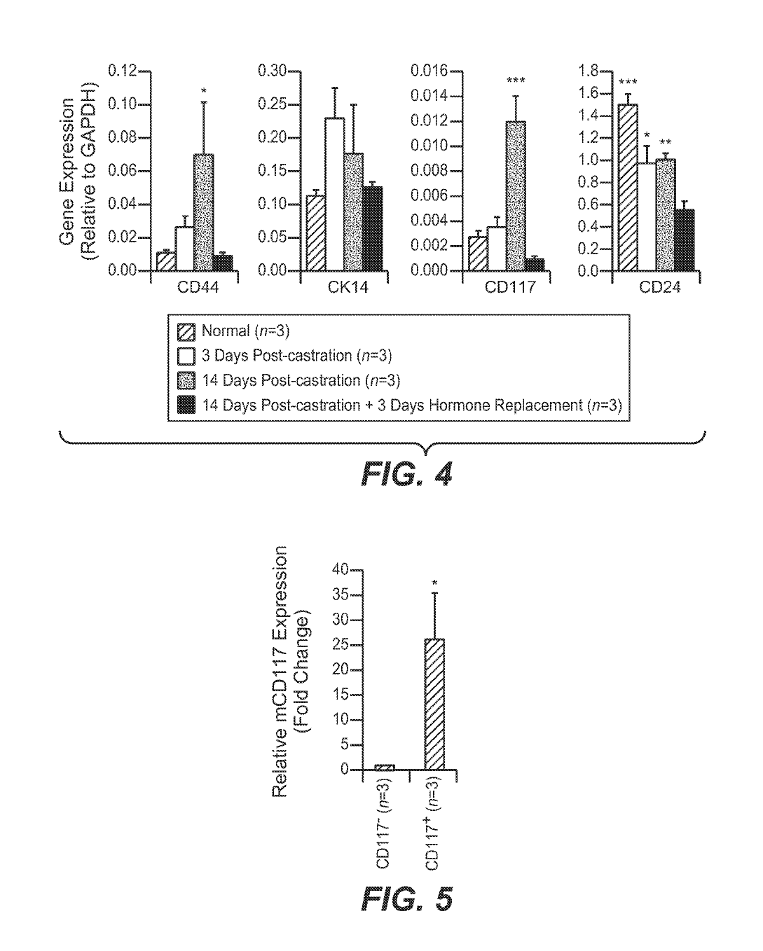

While normal PSCs are androgen-independent and survive the castration process, they remain androgen-responsive and effect prostate regeneration following hormone replacement¹. If normal PSCs expressed CD117, it is expected that CD117 expression would increase following castration (due to stem cell enrichment) and decrease following hormone replacement (due to differentiated cell expansion). Indeed, CD117, CK14, and CD44, but not CD24, exhibited this pattern (FIG. 4). These findings further indicate that CD117 exhibits an expression pattern compatible with that of a normal PSC marker.

### Example 3

### CD117⁺Population is Enriched for PSCs

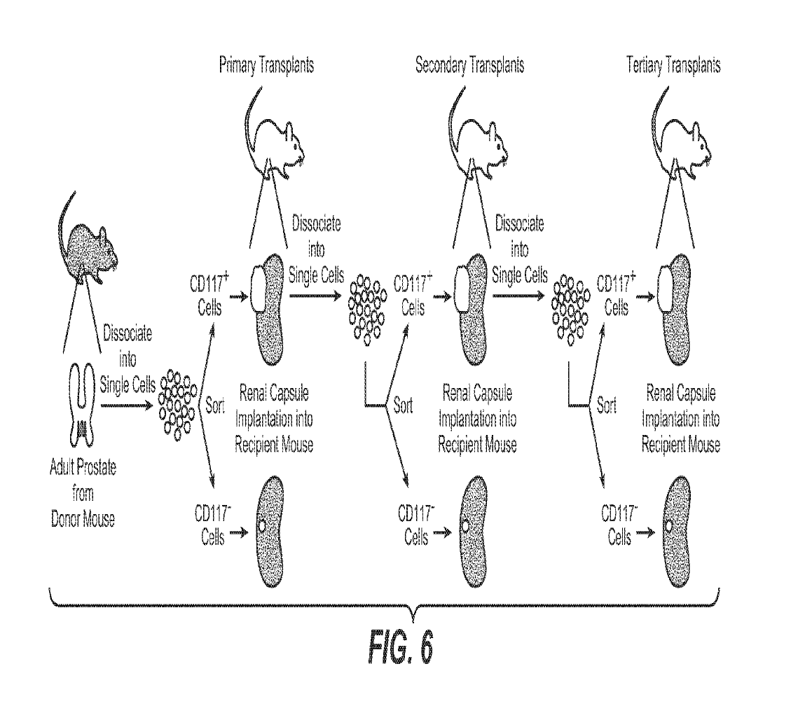

To provide functional evidence that the CD117⁺ population is enriched for PSCs, CD117^{+/-} fractions from dissociated adult C57BL/6 prostates were prepared by magnetic bead sorting and enrichment was confirmed by Q-RT-PCR (FIG. 5). Standard prostate colony formation assays were performed in vitro¹³. CD117⁺ cells, but not CD117⁻ cells, gave rise to numerous lumen-containing colonies. Although this in vitro assay suggests that the CD117⁺ population contains PSCs, the ability of CD117⁺ cells to generate prostates in vivo is an essential assessment of the stem cell phenotype. Using an in vivo prostate generation system^{17,18}, CD117^{+/-} fractions from C57BL/6 mouse donors were combined with rat embryonic urogenital sinus mesenchymal (UGM) stromal cells and implanted under the renal capsule of athymic nu/nu mouse hosts. Although CD117⁻ cells remained viable under the renal capsule, CD117⁻ grafts were small, opaque, and similar to grafts of UGM cells alone in their inability to generate prostates (prostate generation frequency, n=1/10). In contrast, CD117⁺ grafts were large, vascularized, and translucent (prostate generation frequency, n=10/12). Histological examination of CD117⁺ grafts revealed a branching morphology with epithelial tubules composed of basal (CK14) and luminal (CK18) cell lineages. Rare neuroendocrine cells, identified as solitary synaptophysin⁺ cells within the basal compartment of wildtype mouse prostates¹⁹, were observed in several prostatic ducts and acini within the same and across multiple implants. CD117⁺ grafts also expressed the prostate-specific proteins probasin²⁰ and Nkx3.1²¹, indicating functional prostate generation. Using a mouse β1 integrin-specific antibody, it was verified that CD117⁺ grafts were of mouse origin and not due to contaminating rat epithelial cells from the UGM stromal cell preparations. Moreover, it was confirmed that generated prostates were derived from transplanted CD117⁺ donor cells using an MHC class I haplotype H-2k^{b} antibody that specifically recognizes donor (C57BL/6) but not host (athymic nu/nu) mouse cells. These findings demonstrate that the CD117⁺ population is enriched for normal PSCs with functional prostate generation capacity.

### Example 4

### CD117⁺ PSCs have the Ability to Self-Renew

A defining characteristic of stem cells is the ability to self-renew²². To evaluate the self-renewal capacity of CD117⁺ cells and to determine whether reduced numbers of CD117⁺ cells would retain prostate generation capacity, serial transplantations were performed with successively reduced numbers of CD117⁺ cells (FIG. 6). Secondary and tertiary transplants of CD117⁺ cells, but not CD117⁻ cells, gave rise to functional prostates comprises of multiple cell types derived from donor C57BL/6 mouse cells. These findings provide direct evidence that the CD117⁺ population contains normal PSCs with self-renewal capacity.

### Example 5

### CD117⁺ Signaling is Important for Normal Prostate Development and for Prostate Regeneration

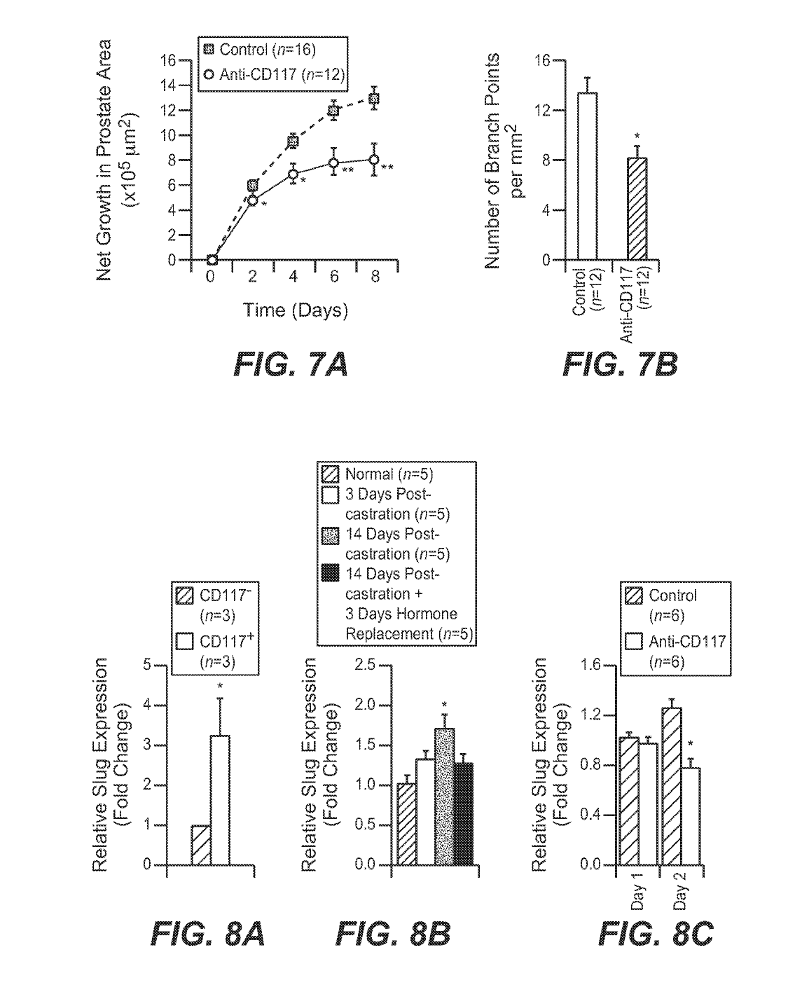

The importance of functional CD117 signaling for normal prostate development was determined Mouse prostrate development begins with epithelial budding from the urogenital sinus at 17.5 days of gestation, with extensive ductal outgrowth and branching occurring during the first three weeks of postnatal development¹¹. Mice homozygous for the dominant-white spotting locus (W/W) lack CD117 signaling and are perinatal lethal²³, thus precluding an assessment of normal prostate development. Heterozygous W/W^{v} mice, which display partially impaired CD117 signaling and hence are viable²³, were analyzed. Despite an equivalent body size at 4 weeks of age, mutant prostates exhibited a reduced size with a similar reduction in adulthood. The proliferation, differentiation, and survival status of mutant prostate cells were examined, since CD117 signaling regulates these processes in various stem cell types²⁴. Mutant prostates displayed inhibited proliferation, although basal/luminal differentiation, cell survival, and vascular/smooth muscle cell recruitment were unaltered compared to wildtype. Similarly, wildtype C57BL/6 prostates harvested at postnatal day 4 and cultured ex vivo in the presence of a function-blocking anti-CD117 antibody (ACK2) displayed inhibited growth and reduced branching (FIGS. 7a and 7b). ACK2 inhibition of CD117 signaling was confirmed by assessing the expression of the transcription factor Slug (FIG. 8a-c), a downstream target of the CD117 pathway²⁵. Notably, treated prostates exhibited attenuated proliferation and an increased basal-to-luminal cell ratio, with no effect on cell survival.

To further evaluate a possible role for CD117 in PSC function in vivo, ACK2 was administered at an in vivo inhibitory dose²⁶ to castrated adult C57BL/6 mice and assessed prostate regeneration following hormone replacement. Similar to ex vivo-treated prostates, attenuating CD117 function in vivo inhibited prostate regeneration concordant with inhibited proliferation and an increased basal-to-luminal cell ratio, with no effect on cell survival or vascular/smooth muscle cell recruitment. These findings indicate that impairment of CD117 signaling with antagonistic blockers, in contrast to partial impairment of CD117 signaling as seen in W/W^{v} mice, may inhibit prostatic luminal cell differentiation, and highlight a potential role for CD117 signaling in normal prostate development. Given that CD117 signaling is important for the function of bone marrow-derived cells (including the mobilization of hematopoietic stem and endothelial progenitor cells²⁷), and that the vasculature and its supporting stroma may play an important role in establishing a PSC niche¹⁴, it is possible that abrogated CD117 function may adversely affect the recruitment/maintenance of non-epithelial cells in the prostate, which in turn may compromise prostate development.

### Example 6

### Stem Cell Marker Frequency

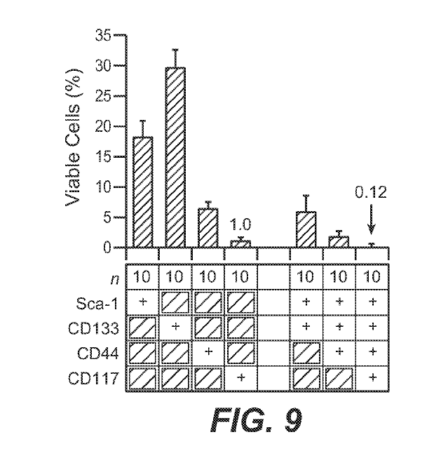

To compare the percentage of CD117⁺ cells in the prostate with that of other PSC populations, lineage-depleted (Lin⁻) population was obtained and flow cytometry was performed. Whereas CD117⁺ cells exhibited the lowest frequency within the viable cell population (approximately 1%), Sca-1⁺ and CD133⁺ cells were detected at much higher frequencies within the viable cell population (FIG. 9). This was consistent with other studies, which have reported Sca-1 and CD133 expression in both stem and non-stem cell types, including stromal and differentiated epithelial cells^{6,28}. These higher frequencies suggest that Sca-1 and CD133 may mark highly heterogeneous subpopulations of prostate cells. Given the heterogeneity of single-stained cell populations, each marker used alone would not be expected to yield a subpopulation composed entirely of stem cells. Therefore combined multiple markers were used to further refine the PSC phenotype. It was determined that Lin⁻Sca-1⁺CD133⁺CD44⁺CD117⁺ cells constituted 0.12% of the viable cell population within the mouse prostate (FIG. 9).

### Example 7

### PSCs with the Phenotype Lin⁻Sca-1⁺CD133⁺CD44⁺CD117⁺ are Capable of Generating a Secretion-Producing Prostate

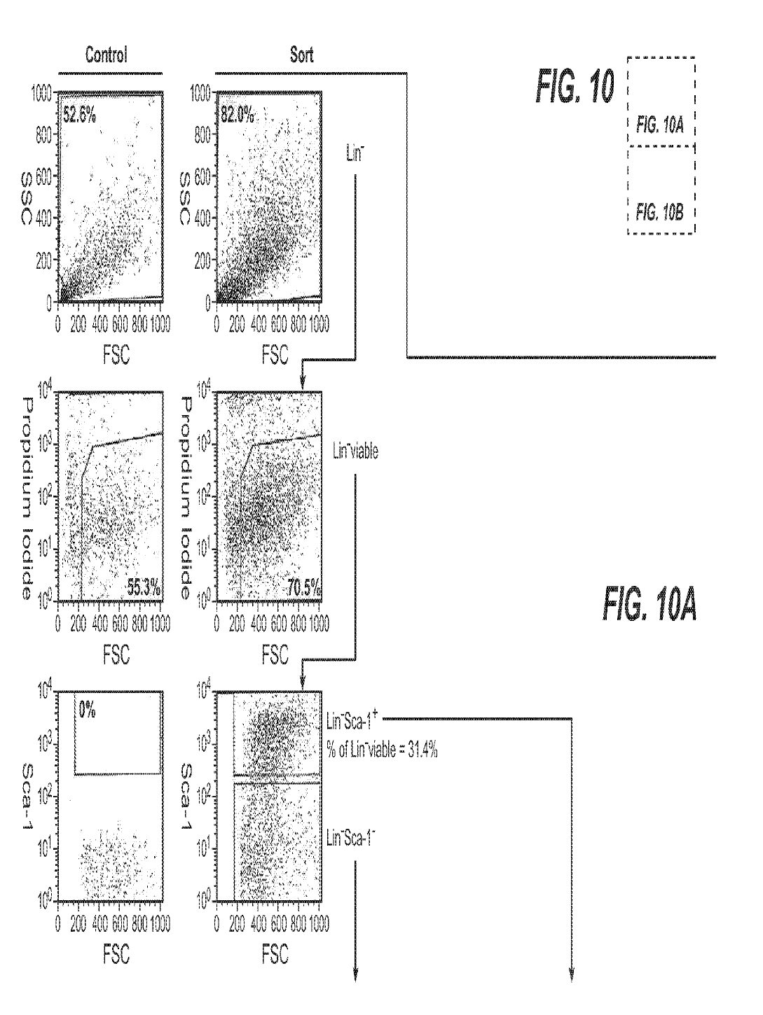

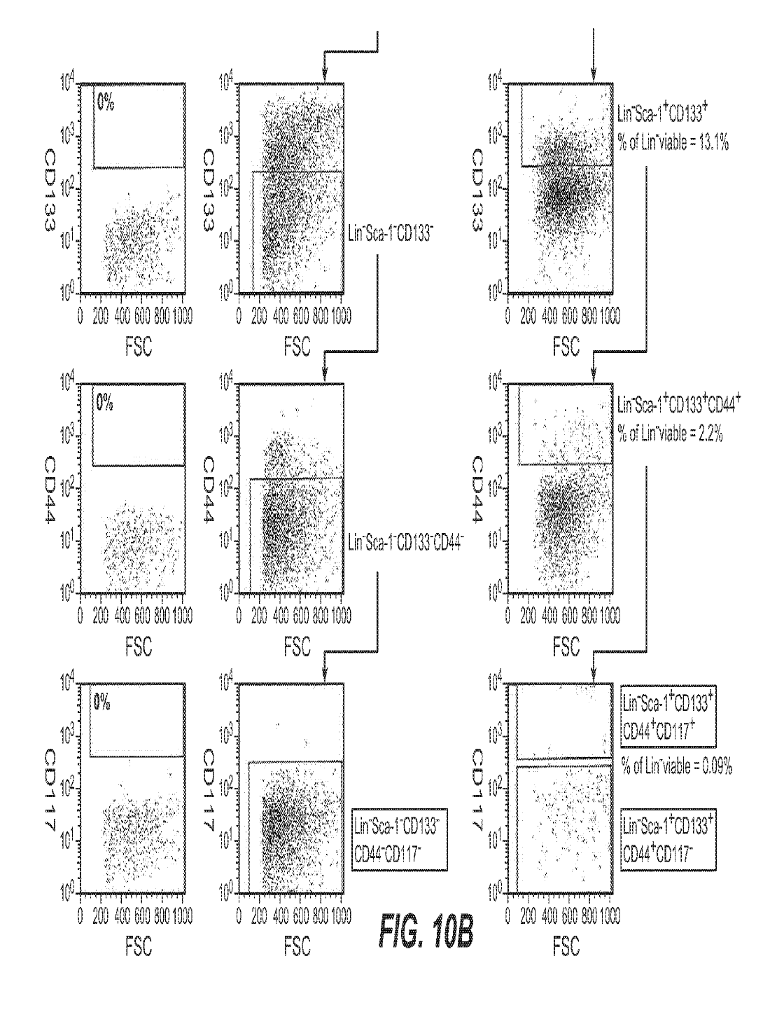

Fluorescence-activated cell sorting (FACS) was performed to obtain cell populations expressing combinations of multiple surface markers (FIG. 10), followed by renal capsule implantation. In this experiment, dissociated prostate cells were sorted by magnetic beads to obtain Lin− cells, which were subsequently sorted by FACS. The Lin− propidium iodide-(Lin−, viable) population was gated on Sca-1 expression to obtain Lin-Sca-1+ and Lin-Sca-1− populations. Sequential gating of the Lin-Sca-1− population for CD133−, CD44−, and CD117− fractions yielded Lin-Sca-1−CD133−CD44−CD117− cells. Sequential gating of the Lin-Sca-1+ population for CD133+ and CD44+ fractions yielded the Lin-Sca-1+CD133+CD44+ population, which was then gated on CD117 expression to obtain Lin-Sca-1+CD133+CD44+CD117+ cells and Lin-Sca-1+CD133+CD44+CD117− cells.

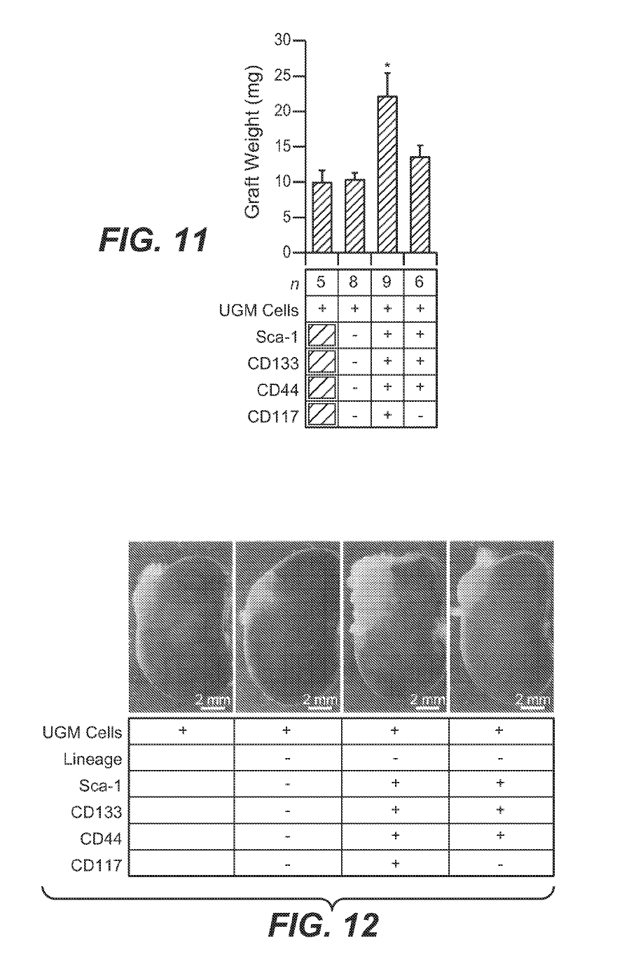

Quantification of graft weight three months after transplantation indicated that only the Lin⁻ Sca-1⁺CD133⁺CD44⁺CD117⁺ population was capable of generating prostates (FIGS. 11 and 12). The prostate generation frequency for the various sorted cell populations were as follows: Lin⁻Sca-1⁻ CD133⁻CD44⁻CD117⁻, n=0/8; Lin⁻Sca-1⁺CD133⁺CD44⁺CD117⁺, n=6/9; Lin⁻Sca-1⁺CD133⁺CD44⁺CD117⁻, n=0/6.

Histological examination was performed (as described in Example 3 above) indicating that the regenerated prostates were composed of basal (CK14) and luminal (CK18) cell lineages and that the prostates expressed the prostate-specific proteins probasin²° and Nkx3.1²¹, indicating functional prostate generation. It was verified using a mouse β1 integrin-specific antibody that the prostates were of mouse origin and not due to contaminating rat epithelial cells from the UGM stromal cell preparations and that the generated prostates were derived from transplanted CD117⁺ donor cells using an MHC class I haplotype H-2k^{b} antibody that specifically recognizes donor (C57BL/6) but not host (athymic nu/nu) mouse cells.

Serial transplantation yielded similar results with the prostate generation frequency for the various sorted cell populations at Lin⁻Sca-1⁻CD133⁻CD44⁻CD117⁻, n=0/3; Lin⁻Sca-1⁺CD133⁺CD44⁺CD117⁺, n=1/3; Lin⁻Sca-1⁺CD133⁺CD44⁺CD117⁻, n=0/3, confirming that the Lin⁻ Sca-1⁺CD133⁺CD44⁺CD117⁺ population contains normal PSCs with self-renewal capacity.

### Example 8

### Prostate Generation from a Single Lin⁻Sca-1⁺CD133⁺CD44⁺CD117⁺ cell

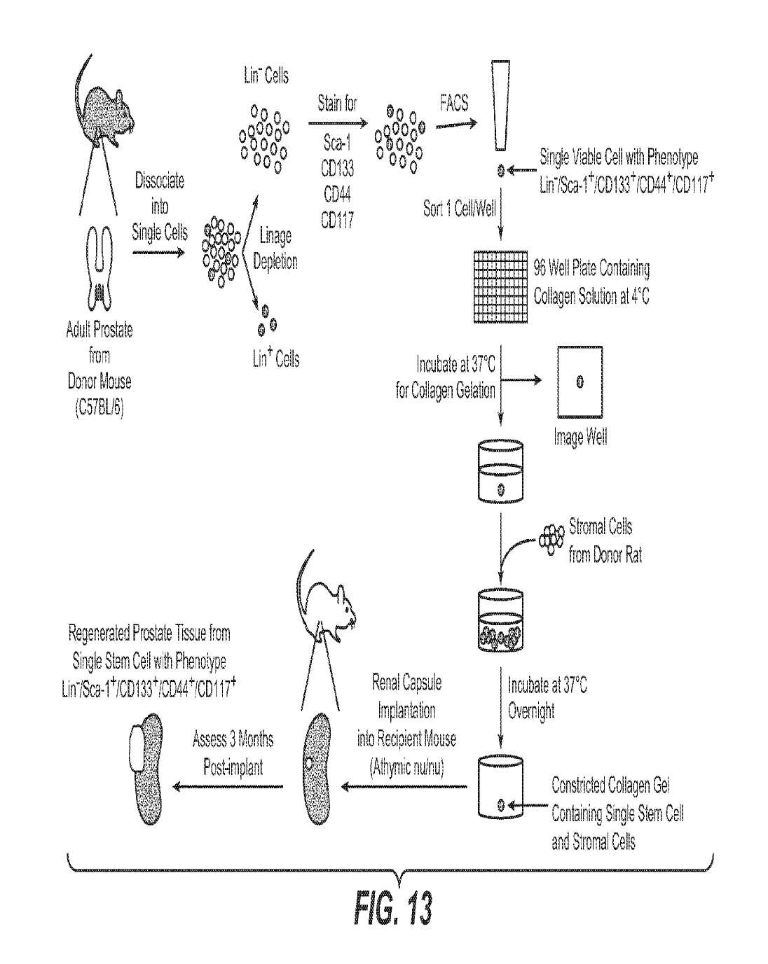

To definitively prove that prostate generation could be achieved from a single Lin⁻Sca-1⁺CD133⁺CD44⁺CD117⁺ cell, single viable cells were sorted into individual wells by FACS. Each well was imaged to confirm the presence of a single cell and single donor C57BL/6 mouse cells were grafted in combination with rat UGM stromal cells under the renal capsule of host athymic nu/nu mice (FIG. 13). More specifically, adult C57BL/6 prostates were harvested, dissociated into single cells, lineage depleted by magnetic bead sorting to obtain the Lin− population, and stained with propidium iodide and antibodies against cell-surface markers Sca-1, CD133, CD44, and CD117. By FACS, single viable (propidium iodide-) Lin-Sca-1+CD133+CD44+CD117+ cells were sorted into individual wells of a 96 well plate containing collagen solution at 4° C. Plates were then microscopically examined in a 37° C. temperature-regulated chamber to confirm the presence of a single cell per well, and digital images of each single cell within each well were captured. Following collagen gelation, rat UGM stromal cells were added to each well (250,000 cells per well) and the plate incubated at 37° C. overnight, resulting in collagen gel constriction. Gels were then grafted under the renal capsule of host athymic nu/nu mice, along with a subcutaneous slow-release testosterone pellet. Three months post grafting, single cell implants were harvested and subjected to histological examination.

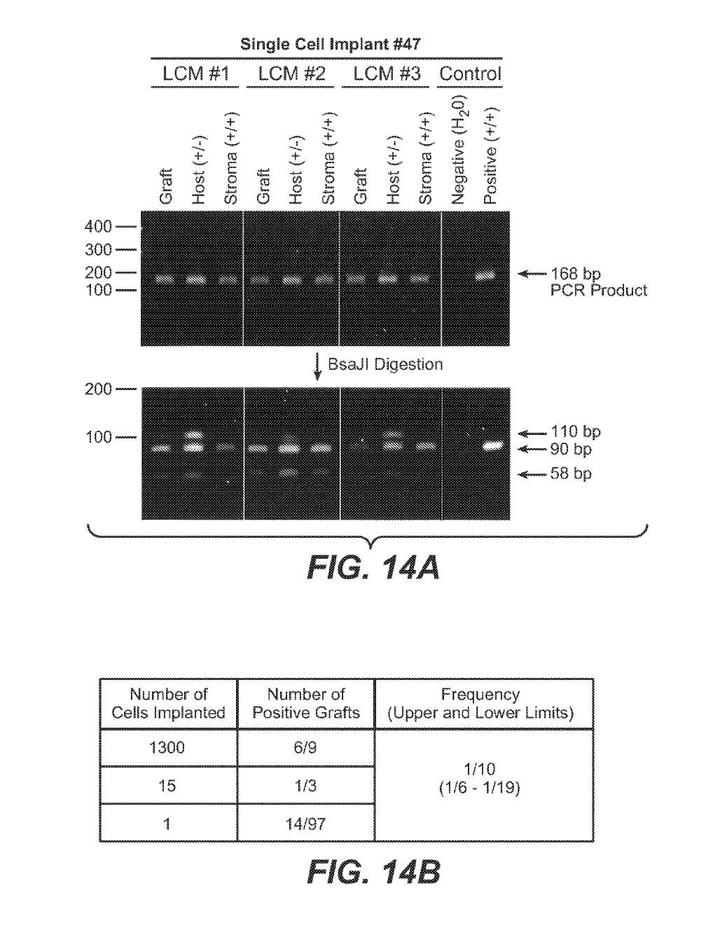

Remarkably, 14 prostates were generated from 97 single cell transplants. Histological analyses confirmed that whereas grafts of UGM cells alone were incapable of prostate generation, grafts of the 14 successful single cell transplants exhibited substantial prostate development. The presence of epithelial tubules comprises of multiple cell lineages and the expression of probasin and Nkx3.1 were confirmed. Importantly, single cell-derived prostates expressed both mouse-specific β1 integrin and C57BL/6 donor-specific H-2k^{b}. It was further confirmed that the generated prostates were derived from a single transplanted donor cell by PCR-based genotyping of laser capture microdissected cells (FIG. 14a). Single cell-derived prostates exhibited an interconnected branching glandular morphology surrounded by a thick layer of stromal cells and connective tissue. By limiting dilution analysis, the frequency of PSCs within the Lin⁻Sca-1⁺CD133⁺CD44⁺CD117⁺ population was determined to be 1 in 10 (FIG. 14b).

### Example 9

### Human PSC

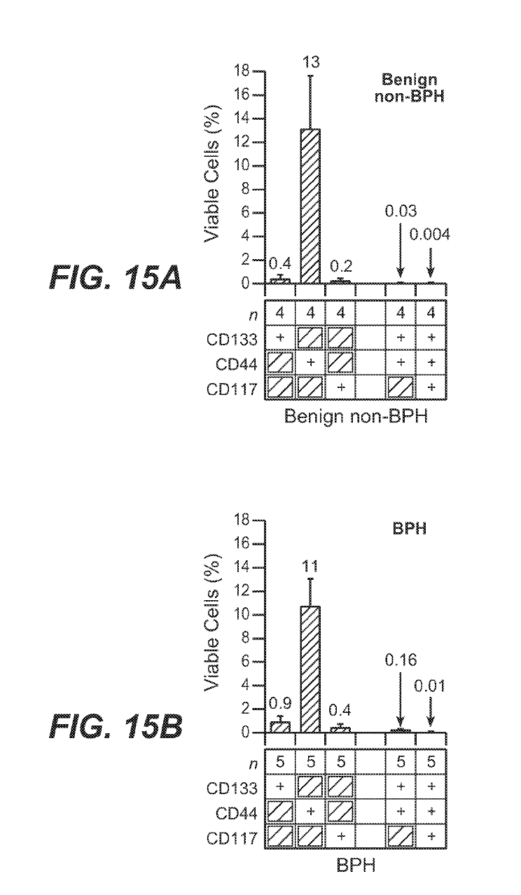

To determine whether CD117 would also mark a potential PSC population within the human prostate, flow cytometric analysis of human benign prostatic hyperplasia (BPH; n=5) and benign non-BPH (n=4) prostate specimens was performed. CD117⁺ cells were observed at a low frequency within the viable cell population in benign non-BPH and BPH specimens (approximately 0.2% and 0.4%, respectively; FIGS. 15a and 15b). A Sca-1 human ortholog has yet to be definitively identified but by combining CD117 with the markers CD133 and CD44, the viable cell frequency of CD133⁺CD44⁺CD117⁺ cells in benign non-BPH and BPH specimens was determined to be 0.004% and 0.01%, respectively (FIGS. 15a and 15b). CD117⁺ cells were detected by immunostaining within the prostate epithelium that co-expressed the basal cell marker p63, in both benign non-BPH and BPH specimens. The benign non-BPH and BPH specimens used for histological analyses were paired specimens taken from the same patient. These findings indicate that CD117 expression, in addition to marking a PSC population within the mouse prostate, is expected to mark a potential PSC population within the human prostate.

All patents, patent applications, patent application publications, and other publications cited or referred to in this specification are herein incorporated by reference to the same extent as if each independent patent, patent application, patent application publication or publication was specifically and individually indicated to be incorporated by reference.

### REFERENCES

1. Isaacs, J. T. Control of Cell Proliferation and Cell Death in the Normal and Neoplastic Prostate. Benign Prostatic Hyperplasia (C. H. Rodgers, D. S. Coffey, G. Cunha, J. T Grayhack, F. Hinman, Jr. and R. Horton, Eds) II, 85-94 (1987).
2. Burger, P. E., et al. Sca-1 expression identifies stem cells in the proximal region of prostatic ducts with high capacity to reconstitute prostatic tissue. Proc Natl Acad Sci USA. 102, 7180-7185 (2005).
3. Liu, A. Y., et al. Cell-cell interaction in prostate gene regulation and cytodifferentiation. Proc Natl Acad Sci USA. 94, 10705-10710 (1997).
4. Richardson, G. D., et al. CD133, a novel marker for human prostatic epithelial stem cells. J Cell Sci. 117, 3539-3545 (2004).
5. Tsujimura, A., et al. Prostatic stem cell marker identified by cDNA microarray in mouse. J Urol. 178, 686-691 (2007).
6. Xin, L., Lawson, D. A. & Witte, O. N. The Sca-1 cell surface marker enriches for a prostate-regenerating cell subpopulation that can initiate prostate tumorigenesis. Proc Natl Acad Sci U S A. 102, 6942-6947 (2005).
7. Abate-Shen, C. & Shen, M. M. Molecular genetics of prostate cancer. Genes Dev. 14, 2410-2434 (2000).
8. Collins, A. T., Habib, F. K., Maitland, N. J. & Neal, D. E. Identification and isolation of human prostate epithelial stem cells based on alpha(2)beta(1)-integrin expression. J Cell Sci. 114, 3865-3872 (2001).
9. Lawson, D. A., Xin, L., Lukacs, R. U., Cheng, D. & Witte, O. N. Isolation and functional characterization of murine prostate stem cells. Proc Natl Acad Sci USA. 104, 181-186 (2007).
10. Schmelz, M., et al. Identification of a stem cell candidate in the normal human prostate gland. Eur J. Cell Biol. 84, 341-354 (2005).
11. Sugimura, Y., Cunha, G. R. & Donjacour, A. A. Morphogenesis of ductal networks in the mouse prostate. Biol Reprod. 34, 961-971 (1986).
12. Salm, S. N., et al. TGF-{beta} maintains dormancy of prostatic stem cells in the proximal region of ducts. J. Cell Biol. 170, 81-90 (2005).
13. Tsujimura, A., et al. Proximal location of mouse prostate epithelial stem cells: a model of prostatic homeostasis. J. Cell Biol. 157, 1257-1265 (2002).
14. Wang, G. M., Kovalenko, B., Wilson, E. L. & Moscatelli, D. Vascular density is highest in the proximal region of the mouse prostate. Prostate. 67, 968-975 (2007).
15. Bonkhoff, H. & Remberger, K. Differentiation pathways and histogenetic aspects of normal and abnormal prostatic growth: a stem cell model. Prostate. 28, 98-106 (1996).
16. Signoretti, S., et al. p63 is a prostate basal cell marker and is required for prostate development. Am J Pathol. 157, 1769-1775 (2000).
17. Cunha, G. R. & Lung, B. The possible influence of temporal factors in androgenic responsiveness of urogenital tissue recombinants from wild-type and androgen-insensitive (Tfm) mice. J Exp Zool. 205, 181-193 (1978).
18. Xin, L., Ide, H., Kim, Y., Dubey, P. & Witte, O. N. In vivo regeneration of murine prostate from dissociated cell populations of postnatal epithelia and urogenital sinus mesenchyme. Proc Natl Acad Sci USA. 100, 11896-11903 (2003).
19. Kurita, T., Medina, R. T., Mills, A. A. & Cunha, G. R. Role of p63 and basal cells in the prostate. Development. 131, 4955-4964 (2004).
20. Johnson, M. A., Hernandez, I., Wei, Y. & Greenberg, N. Isolation and characterization of mouse probasin: An androgen-regulated protein specifically expressed in the differentiated prostate. Prostate. 43, 255-262 (2000).
21. Shen, M. M. & Abate-Shen, C. Roles of the Nkx3.1 homeobox gene in prostate organogenesis and carcinogenesis. Dev Dyn. 228, 767-778 (2003).
22. Weissman, I. L. Stem cells: units of development, units of regeneration, and units in evolution. Cell. 100, 157-168 (2000).
23. Lev, S., Blechman, J. M., Givol, D. & Yarden, Y. Steel factor and c-kit protooncogene: genetic lessons in signal transduction. Crit. Rev Oncog. 5, 141-168 (1994).
24. Ashman, L. K. The biology of stem cell factor and its receptor C-kit. Int J Biochem Cell Biol. 31, 1037-1051 (1999).
25. Perez-Losada, J., et al. Zinc-finger transcription factor Slug contributes to the function of the stem cell factor c-kit signaling pathway. Blood. 100, 1274-1286 (2002).
26. Moss, K. G., Toner, G. C., Chemington, J. M., Mendel, D. B. & Laird, A. D. Hair depigmentation is a biological readout for pharmacological inhibition of KIT in mice and humans. J Pharmacol Exp Ther. 307, 476-480 (2003).
27. Heissig, B., Werb, Z., Rafii, S. & Hattori, K. Role of c-kit/Kit ligand signaling in regulating vasculogenesis. Thromb Haemost. 90, 570-576 (2003).
28. Shmelkov, S. V., et al. CD133 expression is not restricted to stem cells, and both CD133+ and CD133-metastatic colon cancer cells initiate tumors. J Clin Invest. 118, 2111-2120 (2008).
29. Shackleton, M., et al. Generation of a functional mammary gland from a single stem cell. Nature. 439, 84-88 (2006).
30. Stingl, J., et al. Purification and unique properties of mammary epithelial stem cells. Nature. 439, 993-997 (2006).

1.-9. (canceled)

10. A method of isolating a prostate stem cell comprising the steps of:
a. obtaining a lineage depleted (Lin−) prostate cell population.
b. sorting the Lin− cell population to obtain a population of cells that expresses CD117, CD133, and CD44.

11. The method of claim 10, further comprising the step of sorting the Lin− prostate cell population to obtain a population of cells that expresses Sca-1.

12. The method of claim 10 or 11, wherein the cells are sorted using fluorescence-activated cell sorting (FACS).

13. A method of inhibiting the proliferation of a prostate stem cell or prostate cancer stem cell that expresses CD117, CD133, and CD44, comprising contacting the prostate stem cell or prostate cancer stem cell with an therapeutically effective amount of a CD117 antagonist.

14. A method of preventing reoccurrence of prostate cancer in a patient comprising:
a. determining if the patient's prostate cancer comprises a prostate cell that expresses CD117, CD133, and CD44, and
b. administering to the patient a therapeutically effective amount of a CD117 antagonist if the patient's prostate cancer comprises a prostate cell that expresses CD117, CD133, and CD44

15. The method of claim 14, further comprising administering to the patient an effective amount of a CD133 antagonist or a CD44 antagonist.

16.-20. (canceled)

21. A method of providing adjuvant therapy to a patient treated for prostate cancer comprising:
a. determining if the patient's prostate cancer comprises a prostate cell that expresses CD117, CD133, and CD44, and
b. administering to the patient a therapeutically effective amount of a CD117 antagonist if the patient's prostate cancer comprises a prostate cell that expresses CD117, CD133, and CD44.

22. The method of claim 21, further comprising administering to the patient an effective amount of a CD133 antagonist or a CD44 antagonist.

23. The method of claim 14, wherein CD117 antagonist is an anti-CD117 antibody.

24. The method of claim 14, wherein CD117 antagonist is a small molecule.

25. The method of claim 24, wherein the CD117 antagonist is imatinib mesylate or sunitinib malate.

26.-32. (canceled)

33. A method of screening for a compound that inhibits the proliferation of prostate cancer stem cells comprising:
a. contacting a prostate cancer stem cell that expresses CD117, CD133, and CD44 with a test compound, and
b. detecting whether the test compound inhibits proliferation of the cell.
